(19) United States
(12) Patent Application Publication
(10) Pub. No.: US 2025/0222112 A1
(43) Pub. Date: Jul. 10, 2025

(54) RECOMBINANT IMMUNE COMPLEXES TARGETING HERPES SIMPLEX VIRUS

(71) Applicant: ARIZONA BOARD OF REGENTS ON BEHALF OF ARIZONA STATE UNIVERSITY, Scottsdale, AZ (US)

(72) Inventors: Hugh Mason, Phoenix, AZ (US); Mary Pardhe, Phoenix, AZ (US); Andrew Diamos, Tempe, AZ (US); Ian Hogue, Tempe, AZ (US)

(73) Assignee: ARIZONA BOARD OF REGENTS ON BEHALF OF ARIZONA STATE UNIVERSITY, Scottsdale, AZ (US)

(21) Appl. No.: 18/698,931

(22) PCT Filed: Oct. 13, 2023
(86) PCT No.: PCT/US23/76814

§ 371 Date: Apr. 5, 2024

Related U.S. Application Data
(60) Provisional application No. 63/379,483, filed on Oct. 14, 2022.

(51) Int. Cl. A61K 47/42; A61K 47/68; A61P 37/04; C07K 14/005; C07K 16/42

(52) U.S. Cl. CPC A61K 47/42; A61K 47/6873; A61P 37/04; C07K 14/005; C07K 16/42; C12N 2710/16622; C12N 2710/16634; C12N 2710/16671

(57) ABSTRACT

Provided herein is a vaccination composition that comprises a recombinant immune complex (RIC) that includes an immunoglobulin heavy chain; an epitope tag, wherein the immunoglobulin heavy chain binds the epitope tag; and at least a fragment of a herpes simplex virus (HSV) type 2 glycoprotein D (HSV2 glycoprotein D). Related methods, recombinant vectors, and other aspects are also provided.

### CROSS-REFERENCE TO RELATED APPLICATIONS

This application claims the benefit of U.S. Provisional Patent Application Ser. No. 63/379,483, filed Oct. 14, 2022, the disclosure of which is incorporated herein by reference.

### STATEMENT REGARDING FEDERALLY SPONSORED RESEARCH OR DEVELOPMENT

This invention was made with government support under grant U19-A1062150-01 awarded by the National Institutes of Health-National Institute of Allergy and Infectious Diseases. The government has certain rights in the invention.

### REFERENCE TO ELECTRONIC SEQUENCE LISTING

The application contains a Sequence Listing which has been submitted electronically in .XML format and is hereby incorporated by reference in its entirety. Said .XML copy, created on Oct. 13, 2023, is named “0391.0020.xml” and is 22,790 bytes in size. The sequence listing contained in this .XML file is part of the specification and is hereby incorporated by reference herein in its entirety.

### BACKGROUND

The Herpesviridae are a family of enveloped, double-stranded DNA viruses, a number of which cause disease in humans and/or animals. At least seven herpesviruses are associated with infection in humans, including herpes simplex virus type-1 (HSV-1), herpes simplex virus type-2 (HSV-2), varicella zoster virus (VZV), Epstein Barr virus (EBV), cytomegalovirus (CMV), human herpesvirus-6 (HHV-6) and human herpesvirus-7 (HHV-7). Diseases caused by herpesviruses in humans vary from mild to severe, and in some cases, infection is life-threatening.

Vaccines of various types have been proposed for herpesviruses and have included for example, isolated immunogens (e.g., inactivated whole virus particles, viral subunit proteins), live, attenuated virus, and genetically modified viral mutants (e.g., replication-defective viral mutant strains). Live, attenuated or genetically modified viruses do not induce the disease caused by the corresponding wild-type virus in animals or humans but are nonetheless, capable of inducing a specific immune response in such subjects. Replication-defective viral mutant viruses are specifically defective for viral functions that are essential for replication. Such viruses are propagated in complementary cell lines that express the missing viral proteins to allow viral replication. In normal cells, one or more steps in viral replication are blocked, such that normal gene expression within the infected cell is allowed whereas production of progeny virus is not.

Recombinant immune complexes (RICs), fundamentally, are composed of immunoglobulin molecules specific for a desired antigen that are fused to the same antigen that the antibody is specific for. Specifically, the parts of an RIC are an antibody, linked via its C-terminus, to an antigen that is followed by an epitope tag for the antibody. This allows for the binding region of one antibody to bind to the antigen recombinantly fused to another antibody, resulting in the formation of large, highly immunogenic antibody-antigen complexes. RICs can be engineered into ‘universal vaccine platforms’ through the use of antibodies specific for an epitope tag, which allows for the same antibody to be used regardless of the antigen so long as the antibody's corresponding epitope tag is expressed on the antigen (FIG. 1). Thus, RIC can potentiate the immunogenicity of a given antigen.

Accordingly, there is a need for vaccination compositions of use in generating an immune response against herpesviruses.

### SUMMARY

The present disclosure relates, in certain aspects, to vaccination compositions that comprise recombinant immune complexes (RICs) effective at generating immune responses against herpes simplex virus type 2 (HSV2) in mammalian subjects. The present disclosure also provides methods of producing the vaccination compositions in addition to methods of generating an immune response against HSV2 in a mammalian subject. These and other aspects will be apparent upon a complete review of the present disclosure, including the accompanying figures.

In one aspect, the present disclosure provides a vaccination composition that comprises a recombinant immune complex (RIC), comprising: an immunoglobulin heavy chain; an epitope tag (e.g., a 6H epitope tag, etc.), wherein the immunoglobulin heavy chain binds the epitope tag; and at least a fragment of a herpes simplex virus type 2 (HSV2) glycoprotein D (gD).

In some embodiments, the HSV2 gD is a full-length protein. In some embodiments, the epitope tag is linked to the C-terminus of the HSV2. In some embodiments, the HSV2 gD is linked to the C-terminus of the immunoglobulin heavy chain. In some embodiments, the RIC further comprises an immunoglobulin light chain.

In some embodiments, an Immunoglobulin G (IgG) comprises the immunoglobulin heavy chain. In some embodiments, the IgG comprises a human or humanized IgG. In some embodiments, a humanized 6D8 monoclonal antibody comprises the immunoglobulin heavy chain and wherein the epitope tag comprises a 6D8 epitope tag. In some embodiments, the vaccination composition substantially cross-neutralizes herpes simplex virus type 1 (HSV1) and HSV2 when administered to a mammalian subject infected with HSV1 and HSV2.

In one aspect, the present disclosure provides a method of generating an immune response against a herpes simplex virus type 2 (HSV2) in a mammalian subject. The method comprises administering to the mammalian subject a recombinant immune complex (RIC) that comprises: an immunoglobulin heavy chain; an epitope tag, wherein the immunoglobulin heavy chain binds the epitope tag; and at least a fragment of an HSV2 glycoprotein D (gD).

In some embodiments, the HSV2 gD is a full-length protein. In some embodiments, the epitope tag is linked to the C-terminus of the HSV2. In some embodiments, the HSV2 gD is linked to the C-terminus of the immunoglobulin heavy chain. In some embodiments, the RIC further comprises an immunoglobulin light chain. In some embodiments, an Immunoglobulin G (IgG) comprises the immunoglobulin heavy chain. In some embodiments, the IgG comprises a human or humanized IgG. In some embodiments, a humanized 6D8 monoclonal antibody comprises the immunoglobulin heavy chain and wherein the epitope tag comprises a 6D8 epitope tag. In some embodiments, the mammalian subject is infected with herpes simplex virus type 1 (HSV1) and HSV2, and wherein the RIC substantially cross-neutralizes the HSV1 and the HSV2 in the mammalian subject.

In one aspect, the present disclosure provides a recombinant vector comprising a nucleic acid molecule that encodes a recombinant immune complex (RIC) that comprises: an immunoglobulin heavy chain; an epitope tag, wherein the immunoglobulin heavy chain binds the epitope tag; and at least a fragment of a herpes simplex virus type 2 (HSV2) glycoprotein D (gD). In some embodiments, a plasmid comprises the recombinant vector.

### BRIEF DESCRIPTION OF THE DRAWINGS

The accompanying drawings, which are incorporated in and constitute a part of this specification, illustrate certain embodiments, and together with the written description, serve to explain certain principles of the compositions, methods, and related aspects disclosed herein. The description provided herein is better understood when read in conjunction with the accompanying drawings which are included by way of example and not by way of limitation. It will be understood that like reference numerals identify like components throughout the drawings, unless the context indicates otherwise. It will also be understood that some or all of the figures may be schematic representations for purposes of illustration and do not necessarily depict the actual relative sizes or locations of the elements shown.

FIG. 1 depicts an exemplary diagram of universal recombinant immune complex (RIC) targeting an antigen. In this example, the antigen is linked to the C-terminus of the immunoglobulin heavy chain. The epitope tag of one immunoglobulin is bound to the binding site of other immunoglobulins, forming complexes that contain the target antigen.

FIGS. 2A and 2B. Schematic representation of the constructs used in Example 1. (A) Representations of the human IgG1 monoclonal antibody HSV8 and 6-histidine tagged glycoprotein D (gD) from HSV-2. When mixed, HSV8 can bind up to two gD molecules forming an immune complex (IC). (B) The recombinant immune complex (RIC) construct contains the 6D8 monoclonal antibody with C-terminal fusion to gD connected via linker with the epitope tag “e.” This construct forms self-binding clusters via the interaction of the 6D8 variable regions with the epitope tag “e” on another gD-RIC molecule. This interaction results in the formation of larger immune complexes.

FIGS. 3A and 3B. Purification of gD constructs and antibody binding assay. (A) SDS-PAGE and Western blots of the plant-made purified gD constructs. The gD samples were run under reducing conditions, whereas the antibody and RIC constructs were run under nonreducing conditions. The Western blots were probed with either HSV8 (for gD-6H) or H170 (for gD-RIC) to recognize gD, or with an anti-human IgG (H+L) HRP-conjugated antibody. (B) An ELISA was performed to test the binding between plant-made gD and HSV8. Purified gD was bound to the plate, probed with various concentrations of HSV8, and detected with anti-human IgG-HRP conjugate. Points represent mean OD450±standard deviation from two replicates.

FIG. 4. Sucrose gradient density profile of gD-IC and gD-RIC. Using HSV8 and 6D8 mAbs as controls, gD-IC and gD-RIC were separated by sucrose gradient sedimentation using 5/10/15/20/25% discontinuous sucrose layers. The protein concentration of each fraction was analyzed by spectrophotometry and representative results from three independent experiments are shown; direction of sedimentation is left to right. The peak concentration was arbitrarily assigned a value of 1.

FIGS. 5A and 5B. C1q and FcγRIIIa binding. Immune receptor binding ELISA of gD-IC and gD-RIC. ELISA plates were coated with 15 μg/ml (A) human C1q and (B) human FcγRIIIa and incubated with 5-fold serial dilutions of each construct starting at 10 μg/ml, using HSV8 and 6D8 mAbs as monomeric controls. Constructs were detected using polyclonal goat anti-human IgG-HRP. Mean OD450 values from three samples are shown ±standard error. Three stars (***) indicates p<0.001 and one star (*) indicates p<0.05 between the indicated columns using one-way ANOVA with Tukey's post-test for multiple comparisons.

FIGS. 6A and 6B. Mouse immunization and serum titers. BALB/c mice (6 per group) were immunized three times three weeks apart subcutaneously with a dose that would deliver 4 μg gD for each construct or with PBS as a control. Mouse serum samples was collected on days 28, 56, and 86 for doses 1, 2, and 3 respectively. (A) Serially diluted mouse serum was analyzed for total gD-specific IgG production by ELISA. The endpoint was taken as the reciprocal of the greatest dilution that gave an OD₄₅₀ reading at least twice the background. Three stars (***) indicates p<0.001 by one-way ANOVA using Tukey's post-test comparing the indicated columns. (B) Dose 3 mouse serum samples were serially diluted 5-fold starting at a 1:100 dilution and analyzed for IgG2a production by ELISA. Mean OD450 values from three samples are shown ±standard error. Three stars (***) indicates p<0.001 by one-way ANOVA using Tukey's post-test comparing the indicated columns.

FIGS. 7A-7D. Neutralization of HSV-2. Plaque reduction neutralization 50 (PRNT50) assays of (A, B) HSV-2 or (C, D) HSV-1 were performed using serially diluted mouse serum samples from dose 3. Data represent the mean and standard error from four samples for HSV-2, or six samples for HSV-1. Three stars (***) indicates p<0.001 by student's t-test. Representative images showing virus plaques from 3 replicate serum-containing wells and 3 replicate control wells are shown for both HSV-2 and HSV-1.

FIGS. 8A-8D. Proposed Mechanisms of RIC Immune Enhancement. (A) RIC platform enhances complement activation. RICs activate C1q which in turn activates the complement cascade, causing release of C5a and C3a as cleavage products and downstream production of iC3b. Anaphylatoxin release recruits immune cells, including APCs to site of vaccination, which are able to uptake RIC antigen via iC3b:CR2 interactions and Fc:FcR interactions, made more potent via the RIC platform. (B) Enhanced uptake and iC3b production leads to more efficient B cell activation. Presence of iC3b is required for full B cell activation via presentation of antigen from follicular dendritic cells to naïve B cells. If B cells recognize both antigen and iC3b, B cells to migrate to cortical-paracortical junction to gain T cell help. (C) Enhanced DC activation and recruitment leads to more efficient T cell activation. DCs that were recruited to vaccination site travel to LN to activate T cells. Activated T cells travel to cortical: paracortical junction. (D) B cells gain T cell help and can effectively undergo affinity maturation. After getting help from T cells, B cells form germinal centers where they undergo somatic hypermutation and isotype switching.

FIGS. 9A-9G. SDS-PAGE Gels and Westerns from Construct Purifications. Full gel images from each of the SDS-PAGE and western blot experiments used to compile FIG. 2. The lane corresponding to the image used for FIG. 2 is indicated with an asterisk (*). (A) gD-RIC SDS-PAGE gel. (B) gD-6H SDS-PAGE gel. (C) HSV8 SDS-PAGE gel. (D) gD-RIC western blot probed with HSV8. (E) gD-6H western blot probed with HSV8. (F) gD-RIC western blot probed with anti-human IgG. (G) HSV8 western blot probed with anti-human IgG.

FIGS. 10A-10C. SDS-PAGE Gels of Sucrose Gradient Profiles. Fractions 1-9 from sucrose gradients of 6D8, HSV8, gD-IC, and gD-RIC were separated on reducing stain free SDS-PAGE gels. Fraction 1 represents the top of the gradient with the lowest sucrose concentration, while fraction 9 represents the bottom of the gradient with the highest sucrose concentration. Note the increased proportion of gD-RIC in fractions 4-9 compared all other constructs. The leftmost lane contains protein size markers.

FIG. 11. Antibody Response to the Human IgG1 RIC Backbone. ELISA measuring antibody responses using serially diluted (1:100 to 1:10,000) dose 3 serum from mice vaccinated with gD-RIC or PBS. Serum binding was measured using (A) 6D8; (B) 6D8 heavy chain with epitope tag; or (C) gD-RIC containing 6D8, epitope tag, and gD. Representative results from two independent experiments are given as mean OD450 values from two samples ±standard error.

### DEFINITIONS

In order for the present disclosure to be more readily understood, certain terms are first defined below. Additional definitions for the following terms and other terms may be set forth throughout the specification. If a definition of a term set forth below is inconsistent with a definition in an application or patent that is incorporated by reference, the definition set forth in this application should be used to understand the meaning of the term.

As used in this specification and the appended claims, the singular forms “a,” “an,” and “the” include plural references unless the context clearly dictates otherwise. Thus, for example, a reference to “a method” includes one or more methods, and/or steps of the type described herein and/or which will become apparent to those persons skilled in the art upon reading this disclosure and so forth.

It is also to be understood that the terminology used herein is for the purpose of describing particular embodiments only and is not intended to be limiting. Further, unless defined otherwise, all technical and scientific terms used herein have the same meaning as commonly understood by one of ordinary skill in the art to which this disclosure pertains. In describing and claiming the methods, systems, and computer readable media, the following terminology, and grammatical variants thereof, will be used in accordance with the definitions set forth below.

About: As used herein, “about” or “approximately” or “substantially” as applied to one or more values or elements of interest, refers to a value or element that is similar to a stated reference value or element. In certain embodiments, the term “about” or “approximately” or “substantially” refers to a range of values or elements that falls within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value or element unless otherwise stated or otherwise evident from the context (except where such number would exceed 100% of a possible value or element).

Administer: As used herein, “administer” or “administering” a therapeutic agent (e.g., a vaccination composition) to a subject means to give, apply or bring the composition into contact with the subject. Administration can be accomplished by any of a number of routes, including, for example, topical, oral, subcutaneous, intramuscular, intraperitoneal, intravenous, intrathecal and intradermal.

Antibody: As used herein, the term “antibody” refers to an immunoglobulin or an antigen-binding domain thereof. The term includes but is not limited to polyclonal, monoclonal, monospecific, polyspecific, non-specific, humanized, human, canonized, canine, felinized, feline, single-chain, chimeric, synthetic, recombinant, hybrid, mutated, grafted, and in vitro generated antibodies. The antibody can include a constant region, or a portion thereof, such as the kappa, lambda, alpha, gamma, delta, epsilon and mu constant region genes. For example, heavy chain constant regions of the various isotypes can be used, including: IgG1, IgG2, IgG3, IgG4, IgM, IgA₁, IgA₂, IgD, and IgE. By way of example, the light chain constant region can be kappa or lambda. The term “monoclonal antibody” refers to an antibody that displays a single binding specificity and affinity for a particular target, e.g., epitope.

Antigen Binding Portion: As used herein, the term “antigen binding portion” refers to a portion of an antibody that specifically binds to a herpesvirus protein (e.g., a glycoprotein), e.g., a molecule in which one or more immunoglobulin chains is not full length, but which specifically binds to the protein. Examples of binding portions encompassed within the term “antigen-binding portion of an antibody include (i) a Fab fragment, a monovalent fragment consisting of the VLC, VHC, CL and CH1 domains: (ii) a F(ab′)2 fragment, a bivalent fragment comprising two Fab fragments linked by a disulfide bridge at the hinge region; (iii) a Fd fragment consisting of the VHC and CH1 domains; (iv) a Fv fragment consisting of the VLC and VHC domains of a single arm of an antibody, (v) a dAb fragment, which consists of a VHC domain; and (vi) an isolated complementarity determining region (CDR) having sufficient framework to specifically bind, e.g., an antigen binding portion of a variable region. An antigen binding portion of a light chain variable region and an antigen binding portion of a heavy chain variable region, e.g., the two domains of the Fv fragment, VLC and VHC, can be joined, using recombinant methods, by a synthetic linker that enables them to be made as a single protein chain in which the VLC and VHC regions pair to form monovalent molecules (known as single chain Fv (scFV). Such single chain antibodies are also encompassed within the term “antigen binding portion” of an antibody. These antibody portions are obtained using conventional techniques known to those with skill in the art, and the portions are screened for utility in the same manner as are intact antibodies.

Epitope: As used herein, “epitope” refers to the part of an antigen to which an antibody and/or an antigen binding portion binds.

Immune complex: As used herein, the term “immune complex” refers to a complex comprising immunoglobulin molecules or fragments thereof bound to its cognate antigen. As used herein, the term “recombinant immune complex” or “RIC” refers to an immune complex that is not produced by the species that originally produces the immunoglobulin molecule in the immune complex. For example, an exemplary recombinant immune complex comprises human immunoglobulin but is synthesized by plants.

Immune response: As used herein, the term “immune response” refers to a response of a cell of the immune system, such as a B cell, T cell, dendritic cell, macrophage or polymorphonucleocyte, to a stimulus such as an antigen, immunogen, or vaccine. An immune response can include any cell of the body involved in a host defense response, including for example, an epithelial cell that secretes an interferon or a cytokine. An immune response includes, but is not limited to, an innate and/or adaptive immune response. As used herein, a protective immune response refers to an immune response that protects a subject from infection (prevents infection or prevents the development of disease associated with infection). Methods of measuring immune responses are well known in the art and include, for example, measuring proliferation and/or activity of lymphocytes (such as B or T cells), secretion of cytokines or chemokines, inflammation, antibody production and the like. An antibody response or humoral response is an immune response in which antibodies are produced. A “cellular immune response” is one mediated by T cells and/or other white blood cells.

Immunogen: As used herein, the term “immunogen” or “immunogenic” refers to a compound, composition, or substance which is capable, under appropriate conditions, of stimulating an immune response, such as the production of antibodies or a T cell response in an animal, including compositions that are injected or absorbed into an animal. As used herein, “immunize” means to render a subject protected from an infectious disease.

Subject: As used herein, “subject” refers to an animal, such as a mammalian species (e.g., human) or avian (e.g., bird) species. More specifically, a subject can be a vertebrate, e.g., a mammal such as a mouse, a primate, a simian or a human. Animals include farm animals (e.g., production cattle, dairy cattle, poultry, horses, pigs, and the like), sport animals, and companion animals (e.g., pets or support animals). A subject can be a healthy individual, an individual that has or is suspected of having a disease or a predisposition to the disease, or an individual that is in need of therapy or suspected of needing therapy. The terms “individual” or “patient” are intended to be interchangeable with “subject.”

Vaccination: As used herein, the term “vaccination” or “vaccinate” refers to the administration of a composition intended to generate an immune response, for example to a disease-causing agent such as herpesvirus. Vaccination can be administered before, during, and/or after exposure to a disease-causing agent, and/or to the development of one or more symptoms, and in some embodiments, before, during, and/or shortly after exposure to the agent. Vaccines may elicit both prophylactic (preventative) and therapeutic responses. Methods of administration vary according to the vaccine, but may include inoculation, ingestion, inhalation or other forms of administration. Inoculations can be delivered by any of a number of routes, including parenteral, such as intravenous, subcutaneous, intraperitoneal, intradermal, or intramuscular. Vaccines may be administered with an adjuvant to boost the immune response. In some embodiments, vaccination includes multiple administrations, appropriately spaced in time, of a vaccinating composition.

### DETAILED DESCRIPTION

The present disclosure relates, in certain aspects, to recombinant immune complexes (RIC) of use in generating an immune response against herpes virus, such as herpes simplex virus type 2 (HSV2). For example, the present disclosure includes an evaluation of the immunogenic properties of HSV2 glycoprotein D (gD) delivered via traditional immune complex (IC) compared to gD delivered via RIC, and showed the RIC to be substantially better at generating an immune response. The immunogenicity of an IC typically depends strongly on the individual properties of each antibody and the oligomeric nature of its cognate antigen. Therefore, a recombinant immune complex (RIC) system was created which contains, for example, a well-characterized human IgG1 tagged with its own binding site, allowing self-multimerizing immune complexes to be formed with any antigen on a universal platform. Additional details concerning recombinant immune complex (RIC) systems and related aspects are also described in, for example, U.S. patent application Ser. No. 16/404,698, filed May 6, 2019, Ser. No. 16/976,739, filed Aug. 28, 2020, and Ser. No. 17/190,745, filed Mar. 3, 2021, which are each incorporated by references in their entirety.

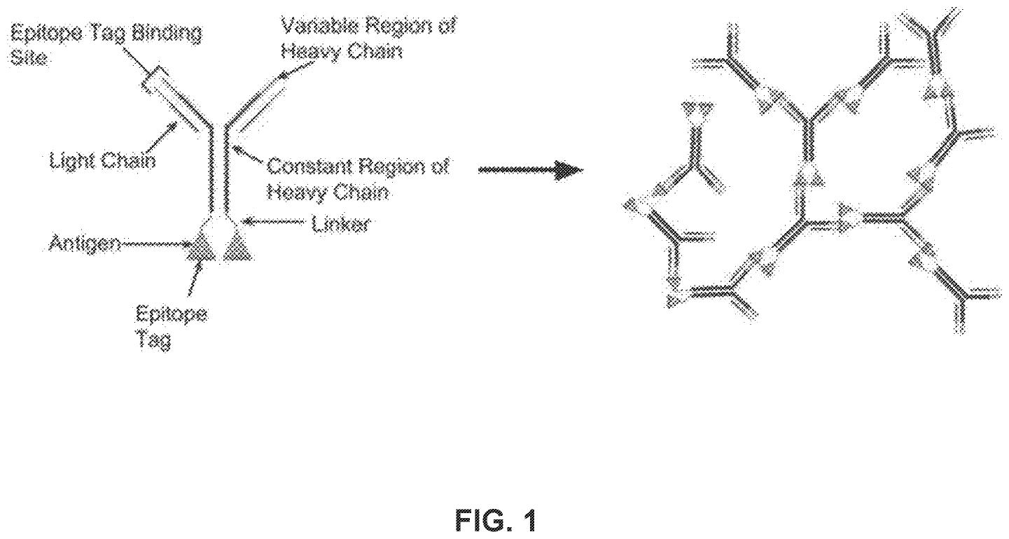

In some embodiments, the RICs described herein comprise an immunoglobulin heavy chain, an epitope tag that can bind to the immunoglobulin heavy chain, and a target antigen. In some aspect, the immunoglobulin heavy chain is a camelid immunoglobulin. In some embodiments, the RIC further comprises an immunoglobulin light chain. Thus, in some aspects, the RIC comprises a standard antibody (two heavy chains and two light chains joined to form a “Y” shaped molecule), an antigen, and an epitope tag that is recognized by the antibody (FIG. 1). The antibody binds to the epitope tags on other antibody fusions and forms a complex. In some embodiments, the RIC comprises human or humanized IgG, and the epitope tag is a 6-histidine (6H or 6-His) epitope tag.

RICs described herein include conventional RICs where the target antigen is linked to the C-terminus of the immunoglobulin heavy chain and the epitope tag is linked to the other end of the target antigen (also referred to herein as “C-RIC”). The recombinant immune complex is produced by fusing a target antigen to the C-terminus of the heavy chain of an immunoglobulin that binds specifically to the antigen, wherein the co-expression of this fusion protein with the light chain of the antibody produces a fully formed immunoglobulin that is self-reactive, and results in the creation of an immune complex due to the bivalent binding capacity of the immunoglobulin. However, antigens with inaccessible N-termini cannot be easily used in the RIC platform without disrupting native antigenic conformation. Also described herein is a design of RICs where the target antigen is linked to the N-terminus of the immunoglobulin heavy chain and the epitope tag is linked to C-terminus of the immunoglobulin heavy chain (also referred to herein as “N-RIC”). In some embodiments, the RICs of the present disclosure are co-administered to subjects along with virus-like particles (VLPs) to produce a greater immune response than can be obtained through delivering either alone at the same does of the antigen.

In certain embodiment of an expression vector encoding RICs, the expression vector comprises a expression cassette encoding the immunoglobulin heavy chain, the target antigen, and the epitope tag. In some aspects, the expression vector further comprises a second expression cassette encoding the immunoglobulin light chain. These and other attributes of the present disclosure will be apparent upon a complete review of the specification, including the accompanying figures.

In some embodiments, a RIC or a component thereof of the present disclosure is encoded by a synthetic polynucleotide that comprises one or more of the nucleotide sequences (or complements thereof) of SEQ ID. NOS: 1-8 (shown below in Table 1) or comprises a polynucleotide having at least 80%, 85%, 90%, 95%, 99% sequence identity with one or more of SEQ ID NOS: 1-8. The phrase “functional portion, fragment, or variant thereof” in the context of the proteins described herein, refers to a portion or fragment of the full-length protein or a non-wild-type form of the protein (full-length, portion, or fragment thereof) that retains a desired property or function, such as improving immunogenicity, effectuating vaccination, or the like. In some embodiments, vaccine compositions of the present disclosure comprise RICs that comprise an HSV2-gD protein, or a functional portion, or fragment or variant thereof.

TABLE 1

| | SEQ | |
|---|---|---|
| DNA SEQUENCE (5′→3′) | ID NO | Description |
| ATGGGATGGTCTTGCATCATACTOTTTCTTGTTGCAACTGCTACAGGTGTCCACTCT | 1 | IgG heavy |
| | | chain |
| | | signal |
| | | peptide |
| GATGTTCAGCTTCTOGAGTCTGGAGGTGGTCTTGTGCAACCTGGAGGTTCCTTGAGACTCTC | 2 | IgG1 |
| CTGTGCAGCTTCAGGGTTTGACTTCAGTAGGTACTGGATGAGTTGGGTTCGTCAAGCTCCTG | | heavy |
| GGAAAGGACTAGAATGGATTGGAGAGATCAATCCAGATTCAAGTACCATCAACTATACTOCAT | | chain |
| CTCTGAAGGATCGCTTCACCATTTCCAGAGACAATGCCAAGAACACGTTGTATOTTCAGATGA | | |
| ACAGCTTGAGGACTGAAGACACAGCCTTGTACTACTGCACAAGACAGGGCTATGGCTACAAC | | |
| TACTGGGGTCAAGGCACCACTGTCACAGTGTCTTCAGCTAGCACCAAAGGTCCATOGGTCTT | | |
| TCCACTGGCACCTTCTTCCAAGAGTACTTCTGGAGGCACAGCTGCACTGGGTTGTCTTGTCA | | |
| AGGACTACTTTCCAGAACCTGTTACGGTTTCGTGGAACTCAGGTGOTCTGACCAGTGGAGTG | | |
| CACACCTTTCCAGCTGTTCTTCAGTCCTCAGGATTGTATTCTCTTAGCAGTGTTGTGACTGTTC | | |
| CATCCTCAAGCTTGGGCACTCAGACCTACATCTGCAATGTGAATCACAAACCCAGCAACACCA | | |
| AGGTTGACAAGAAAGTTGAGCCCAAGTCTTGTGACAAGACTCATACGTGTCCACCGTGCCCA | | |
| GCACCTGAACTTCTTGGAGGACCGTCAGTCTTCTTGTTTCCTCCAAAGCCTAAGGATACCTTG | | |
| ATGATCTCCAGGACTCCTGAAGTCACATGTGTAGTTGTGGATGTGAGCCATGAAGATCCTGA | | |
| GGTGAAGTTCAACTGGTATGTGGATGGTGTGGAAGTGCACAATGCCAAGACAAAGCCGAGAG | | |
| AGGAACAGTACAACAGCACGTACAGGGTTGTCTCAGTTCTCACTGTTCTOCATCAAGATTGGT | | |
| TGAATGGCAAAGAGTACAAGTGCAAGGTCTCCAACAAAGCCCTCCCAGCCCCCATTGAGAAG | | |
| ACCATTTCCAAAGCGAAAGGGCAACCCCGTGAACCACAAGTGTACACACTTCCTCCATCTCG | | |
| CGATGAACTGACCAAGAACCAGGTCAGCTTGACTTGCCTGGTGAAAGGCTTCTATCCCTOTG | | |
| ACATAGCTGTAGAGTGGGAGAGCAATGGGCAACCGGAGAACAACTACAAGACTACACCTCCC | | |
| GTTCTCGATTOTGACGGOTCCTTCTTCCTCTACAGCAAGCTCACAGTGGACAAGAGCAGGTG | | |
| GCAACAAGGGAATGTCTTCTCATGCTCCGTGATGCATGAGGCTCTTCACAATCACTACACACA | | |
| GAAGAGTCTCTCCTTGTCTCCGGGTAAA | | |
| GGAGGTGGOGGATCAGGTGGAGGCGGTTCAGGCGGAGGTGGATCC | 3 | Linker |
| AAATATGCATTAGCTGATCCTAGTOTTAAAATGGCAGATOCTAATAGATTCAGAGGAAAGAATC | 4 | HSV |
| TTCCAGTTTTGGACCAACTTACAGATCCTCCAGGAGTCAAAAGAGTTTATCATATTCAACCTTC | | gD306 |
| TCTTGAGGACCCTTTCCAACCTCCTAGCATACCTATAACTGTTTATTACGCTGTGOTTGAAAG | | |
| GGCTTGTAGGTCTGTGCTCCTACATGCACCATCTGAGGOTCCTCAGATAGTTAGAGGTGOTT | | |
| CTGATGAAGCAAGGAAACATACTTACAATTTGACCATTGOTTGGTATAGGATGGGAGACAATT | | |
| GTGCTATCCCTATTACCGTTATGGAGTACACTGAATGTCCCTACAACAAGTCTTTGGGAGTTT | | |
| GCCCTATTAGAACTCAGCCAAGGTGGAGTTACTATGATTCCTTCTCTGCAGTTTCAGAAGATA | | |
| ATTTGGGATTCTTGATGCATGCTCCTGCCTTTGAGACAGCTGGCACATACTTGAGGCTTGTGA | | |
| AGATTAACGACTGGACTGAGATTACACAATTCATACTTGAACACAGGGCCAGAGCATCCTGCA | | |
| AGTATGCTCTCCCACTTAGGATTCCTCCAGCTGCATGCCTCACCTCAAAAGOTTATCAACAGG | | |
| GTGTGACTGTCGATAGTATTGGCATGTTACCTOGTTTTATOCCAGAGAACCAGAGAACCGTTG | | |
| CATTGTACTCACTTAAGATCGCAGGATGGCACGGTCCAAAGCCTCCATACACTTCAACTTTGC | | |
| TTCCACCTGAGTTGTCTGACACAACTAACGCTACACAACCTGAACTCGTTCCAGAAGACCCAG | | |
| AAGATTCTGCCCTOTTAGAAGATCCAGCTGGCACTGTGTCTTCACAAATTCCTCCAAACTGGC | | |
| ACATTOCATCAATTCAGGATGTTGCTCCACACCAt | | |
| ACTAGT | 5 | SpeI linker |
| CATAACACTCCTGTTTACAAGCTGGACATATCTGAGGCAACTCAA | 6 | Epitope |
| | | tag |
| ATGGGATGGTCTTGCATCATTCTCTTCTTGGTAGCCACAGCTACAGGTGTCCACTCC | 7 | IgG light |
| | | chain |
| | | signal |
| | | peptide |
| GATGTTTTGATGACTCAAAGCCCTCTCTCACTTCCTGTGACTCTTGGACAGCCCGCATCCATA | 8 | IgG light |
| TOTTGCAGATCTAGTCAGAGTATTGTTCATAGTAACGGCAACACCTACTTGGAATGGTATCTG | | chain |
| CAGAAACCAGGCCAGTCTOCAAAGCTTCTGATCTACAAGGCTTCCAATCGTTTCTOTGGTGTC | | |
| CCAGACAGGTTTAGTGGCAGTGGATCAGGGACTGACTTCACATTGAAGATCAGCAGAGTTGA | | |
| GGCTGAAGATGCGGGAGTGTACTATTGTCTTCAAGGTTCACATGTTCCGTCAACGTTTGGAG | | |
| GTGGGACCAAAGTGGAGATCAAGACTGTTGCGGCGCCATCTGTOTTCATOTTTCCTCCATCT | | |
| GATGAACAACTCAAGTCTGGAACTGOTTCTGTTGTGTGCCTTCTGAACAACTTOTATCCTAGA | | |
| GAAGCCAAAGTACAGTGGAAGGTTGACAATGCTCTTCAATCAGGTAACTCCCAGGAGAGTGT | | |
| CACAGAGCAAGATTCCAAGGATTCCACCTACAGCCTCTCAAGTACCTTGACGTTGAGCAAGG | | |
| CAGACTATGAGAAACACAAAGTGTACGCATGCGAAGTCACTCATCAGGGOCTGTCATCACCC | | |
| GTGACAAAGAGCTTCAACAGGGGAGAGTGT | | |

In some embodiments, a RIC of the present disclosure comprises a polypeptide comprising one or more of the amino acid sequences of SEQ ID. NO: 9-14 (shown below in Table 2) or comprises a polypeptide having at least 80%, 85%, 90%, 95%, 99% sequence identity with one or more of SEQ ID NOS: 9-14.

TABLE 2

| | SEQ | |
|---|---|---|
| AMINO ACID SEQUENCE | ID NO | Description |
| MGWSCIILFLVATATGVHS | 9 | IgG heavy |
| | | chain |
| | | signal |
| | | peptide |
| DVQLLESGGGLVQPGGSLRLSCAASGFDFSRYWMSWVRQAPGKGLEWIGEINPDSSTINYTPSL | 10 | IgG heavy |
| KDRFTISRDNAKNTLYLQMNSLRTEDTALYYCTRQGYGYNYWGQGTTVTVSSASTKGPSVFPLA | | chain |
| PSSKSTSGGTAALGQLVKDYFPEPVTVSWNSGALTSGVHTFPAVLQSSGLYSLSSVVTVPSSSLG | | |
| TQTYICNVNHKPSNTKVDKKVEPKSCDKTHTCPPCPAPELLGGPSVFLFPPKPKDTLMISRTPEVT | | |
| CVVVDVSHEDPEVKFNWYVDGVEVHNAKTKPREEQYNSTYRVVSVLTVLHQDWLNGKEYKCKV | | |
| SNKALPAPIEKTISKAKGQPREPQVYTLPPSRDELTKNQVSLTCLVKGFYPSDIAVEWESNGQPEN | | |
| NYKTTPPVLDSDGSFFLYSKLTVDKSRWQQGNVFSCSVMHEALHNHYTQKSLSLSPGK | | |
| GGGGSGGGGSGGGGS | 11 | Linker |
| KYALADPSLKMADPNRFRGKNLPVLDQLTDPPGVKRVYHIQPSLEDPFQPPSIPITVYYAVLERAC | 12 | HSV |
| RSVLLHAPSEAPQIVRGASDEARKHTYNLTIAWYRMGDNCAIPITVMEYTECPYNKSLGVCPIRTQ | | gD306 |
| PRWSYYDSFSAVSEDNLGFLMHAPAFETAGTYLRLVKINDWTEITQFILEHRARASCKYALPLRIP | | |
| PAACLTSKAYQQGVTVDSIGMLPRFIPENQRTVALYSLKIAGWHGPKPPYTSTLLPPELSDTTNAT | | |
| QPELVPEDPEDSALLEDPAGTVSSQIPPNWHIPSIQDVAPHH | | |
| TS | 13 | SpeI linker |
| HNTPVYKLDISEATQ | 14 | Epitope |
| | | tag |

A synthetic polynucleotide encoding a RIC, or component thereof, of the present disclosure can be comprised within an expression cassette. The term “expression cassette” or “expression vector” as used herein refers to a nucleotide sequence, which is capable of affecting expression of a protein coding sequence in a host compatible with such sequences. Expression cassettes typically include at least a promoter operably linked with the polypeptide coding sequence; and, optionally, with other sequences, e.g., transcription termination signals. Additional factors necessary or helpful in effecting expression may also be included, e.g., enhancers. “Operably linked”, refers to linkage of a promoter upstream from a DNA sequence such that the promoter mediates transcription of the DNA sequence. Thus, expression cassettes include plasmids, recombinant viruses, any form of a recombinant “naked DNA” vector, and the like. In some embodiments, expression cassettes include elements that have been codon optimized for expression in the intended host.

The term “immunogen” or “immunogenic composition” is synonymous with “antigen or antigenic” and refers to a compound or composition comprising a peptide, polypeptide or protein which is “immunogenic,” i.e., capable of eliciting, augmenting or boosting a cellular and/or humoral immune response, either alone or in combination or linked or fused to another substance. An immunogenic composition can be a peptide of at least about 5 amino acids, a peptide of 10 amino acids in length, a fragment 15 amino acids in length, a fragment 20 amino acids in length or greater; smaller immunogens may require presence of a “carrier” polypeptide e.g., as a fusion protein, aggregate, conjugate or mixture, preferably linked (chemically or otherwise) to the immunogen. The immunogen can be recombinantly expressed from a vaccine vector, which can be naked DNA comprising the immunogen's coding sequence operably linked to a promoter, e.g., an expression cassette. The immunogen includes one or more antigenic determinants or epitopes, which may vary in size from about 3 to about 15 amino acids. In some embodiments, the immunogen or antigen is a polypeptide comprising an HSV2-gD protein as described herein.

In accordance with some embodiments, the present disclosure provides a recombinant vector encoding the RIC vaccine compositions described herein. By “nucleic acid” as used herein includes “polynucleotide,” “oligonucleotide,” and “nucleic acid molecule,” and generally means a polymer of DNA or RNA, which can be single-stranded or double-stranded, synthesized or obtained (e.g., isolated and/or purified) from natural sources, which can contain natural, non-natural or altered nucleotides, and which can contain a natural, non-natural or altered internucleotide linkage, such as a phosphoroamidate linkage or a phosphorothioate linkage, instead of the phosphodiester found between the nucleotides of an unmodified oligonucleotide. It is generally preferred that the nucleic acid does not comprise any insertions, deletions, inversions, and/or substitutions. However, it may be suitable in some instances, as discussed herein, for the nucleic acid to comprise one or more insertions, deletions, inversions, and/or substitutions, such as when a given polynucleotide encodes a functional portion, fragment, or variant of an HSV2-gD protein.

Preferably, the nucleic acids of the present disclosure are recombinant. As used herein, the term “recombinant” refers to (i) molecules that are constructed outside living cells by joining natural or synthetic nucleic acid segments to nucleic acid molecules that can replicate in a living cell, or (ii) molecules that result from the replication of those described in (i) above. For purposes herein, the replication can be in vitro replication or in vivo replication.

The nucleic acids can be constructed based on chemical synthesis and/or enzymatic ligation reactions using procedures known in the art. For example, a nucleic acid can be chemically synthesized using naturally occurring nucleotides or variously modified nucleotides designed to increase the biological stability of the molecules or to increase the physical stability of the duplex formed upon hybridization (e.g., phosphorothioate derivatives and acridine-substituted nucleotides). Examples of modified nucleotides that can be used to generate the nucleic acids include, but are not limited to, 5-fluorouracil, 5-bromouracil, 5-chlorouracil, 5-iodouracil, hypoxanthine, xanthine, 4-acetylcytosine, 5-(carboxyhydroxymethyl) uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-substituted adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-N6-isopentenyladenine, uracil-5-oxyacetic acid, wybutoxosine, pseudouracil, queosine, 2-thiocytosine, 5-methyl-2-thiouracil, 2-thiouracil, 4-thiouracil, 5-methyluracil, uracil-5-oxyacetic acid methylester, 3-(3-amino-3-N-2-carboxypropyl) uracil, and 2,6-diaminopurine. Alternatively, one or more of the nucleic acids of the invention can be purchased from companies, such as Macromolecular Resources (Fort Collins, CO) and Synthegen (Houston, TX).

In some embodiments, the substituted nucleic acid sequence may be optimized. Without being bound to a particular theory, it is believed that optimization of the nucleic acid sequence increases the translation efficiency of the mRNA transcripts. Optimization of the nucleic acid sequence may involve substituting a native codon for another codon that encodes the same amino acid, but can be translated by tRNA that is more readily available within a cell, thus increasing translation efficiency. Optimization of the nucleic acid sequence may also reduce secondary mRNA structures that would interfere with translation, thus increasing translation efficiency. In some embodiments, codon optimization is performed using Genscript.

In some embodiments, the present disclosure also provides an isolated or purified nucleic acid comprising a nucleotide sequence which is complementary to the nucleotide sequence of any of the nucleic acids described herein or a nucleotide sequence which hybridizes under stringent conditions to the nucleotide sequence of any of the nucleic acids described herein.

The nucleic acids of the present disclosure can be incorporated into a recombinant expression vector. In this regard, the invention provides recombinant expression vectors comprising any of the nucleic acids of the invention. For purposes herein, the term “recombinant expression vector” means a genetically-modified oligonucleotide or polynucleotide construct that permits the expression of an mRNA, protein, polypeptide, or peptide by a host cell, when the construct comprises a nucleotide sequence encoding the mRNA, protein, polypeptide, or peptide, and the vector is contacted with the cell under conditions sufficient to have the mRNA, protein, polypeptide, or peptide expressed within the cell. The vectors of the present disclosure are not naturally occurring as a whole. However, parts of the vectors can be naturally occurring. The recombinant expression vectors can comprise any type of nucleotides, including, but not limited to DNA and RNA, which can be single-stranded or double-stranded, synthesized or obtained in part from natural sources, and which can contain natural, non-natural or altered nucleotides. The recombinant expression vectors can comprise naturally occurring, non-naturally occurring internucleotide linkages, or both types of linkages. Preferably, the non-naturally occurring or altered nucleotides or internucleotide linkages does not hinder the transcription or replication of the vector.

In some embodiments, the expression cassette encoding a RIC or a component thereof will be inserted into a DNA vector or plasmid. The recombinant expression vector of the present disclosure can be any suitable recombinant expression vector, and can be used to transform or transfect any suitable host. Suitable vectors include those designed for propagation and expansion or for expression or both, such as plasmids and viruses. The vector can be selected from the group consisting of the pSectag2B or pVAX1 series (ThermoFisher Scientific, Carlsbad, CA), the pUC series (Fermentas Life Sciences), the pBluescript series (Stratagene, LaJolla, CA), the pET series (Novagen, Madison, WI), the pGEX series (Pharmacia Biotech, Uppsala, Sweden), pcDNA3 family of plasmids, the pNGVL4a plasmid, and the pEX series (Clontech, Palo Alto, CA). Bacteriophage vectors, such as λGT10, λGT11, λZapII (Stratagene), λEMBL4, and λNM1149, also can be used. Examples of plant expression vectors include pBI01, pBI101.2, pBI101.3, pBI121 and pBIN19 (Clontech). Examples of animal expression vectors include pEUK-CI, pMAM and pMAMneo (Clontech). The recombinant expression vectors of the present disclosure can be prepared using standard recombinant DNA techniques well known to persons having ordinary skill in the art. Constructs of expression vectors, which are circular or linear, can be prepared to contain a replication system functional in a prokaryotic or eukaryotic host cell. Replication systems can be derived, e.g., from ColEI, 2μ plasmid, λ, SV40, bovine papilloma virus, and the like. Additional expression vectors are disclosed herein, including in Example 1.

Desirably, the recombinant expression vector comprises regulatory sequences, such as transcription and translation initiation and termination codons, which are specific to the type of host (e.g., mammalian, bacterium, fungus, plant, or animal) into which the vector is to be introduced, as appropriate and taking into consideration whether the vector is DNA- or RNA-based.

The recombinant expression vector can include one or more marker genes, which allow for selection of transformed or transfected hosts. Marker genes include biocide resistance, e.g., resistance to antibiotics, heavy metals, etc., complementation in an auxotrophic host to provide prototrophy, and the like. Suitable marker genes for the expression vectors disclosed herein may include, for instance, neomycin/G418 resistance genes, hygromycin resistance genes, histidinol resistance genes, tetracycline resistance genes, and ampicillin resistance genes, among others.

The recombinant expression vector can include one or more marker genes, which allow for selection of transformed or transfected hosts. Marker genes include biocide resistance, e.g., resistance to antibiotics, heavy metals, etc., complementation in an auxotrophic host to provide prototrophy, and the like. Suitable marker genes for the expression vectors disclosed herein may include, for instance, neomycin/G418 resistance genes, hygromycin resistance genes, histidinol resistance genes, tetracycline resistance genes, and ampicillin resistance genes, among others.

Recombinant expression vectors can comprise a native or nonnative promoter operably linked to the nucleotide sequence encoding the RICs or components thereof, or to the nucleotide sequence which is complementary to or which hybridizes to the nucleotide sequence encoding the RICs or components thereof. The selection of promoters, e.g., strong, weak, inducible, tissue-specific and developmental-specific, is within the ordinary skill of the artisan. Similarly, the combining of a nucleotide sequence with a promoter is also within the skill of the artisan. The promoter can be a non-viral promoter or a viral promoter, e.g., a cytomegalovirus (CMV) promoter, an SV40 promoter, an RSV promoter, and a promoter found in the long-terminal repeat of the murine stem cell virus.

In accordance with some embodiments, the present disclosure provides various pharmaceutical compositions comprising the RICs described herein for use as a vaccine. Thus, in further embodiments, the present disclosure provides the use of a pharmaceutical composition comprising a vaccine, and a pharmaceutically acceptable carrier, as a medicament, preferably as a medicament for the treatment of a herpes viral infection in a subject.

In a further embodiment, the present invention provides a method for treating a herpes viral infection in a subject in need thereof comprising administering to the subject an effective amount of the vaccine compositions described herein.

In some embodiments, the present disclosure provides methods of providing prophylaxis to, and/or treating a herpes viral infection in, a subject in need thereof comprising administering to the subject an effective amount of a composition disclosed herein. In some embodiments, the composition is administered to the subject prior to, concurrent with, and/or after administering at least one antiviral agent to the subject. In some embodiments, the composition is administered as one or more boost doses after an initial administration of the composition to the subject.

In some embodiments, the term “administering” means that the compositions of the present disclosure are introduced into a subject, preferably a subject receiving treatment for a herpes viral infection, and the compounds are allowed to come in contact with the one or more infected cells or population of cells in vivo. In some embodiments, the composition is administered intramuscularly and/or intranasally to the subject.

It will be understood to persons having ordinary skill in the art that the vaccine compositions described herein can be administered in a regimen where there is a first or priming dose of vaccine composition administered to the subject, then after a period of time (e.g., 5 to 180 or more days), a second, third or more boost dose of vaccine is then administered to the subject. In some embodiments, the boost dose is administered 5, 6, 7, 8, 9, 10, 15, 20, 30, 40 up to 50 days apart.

In some embodiments, the carrier is a pharmaceutically acceptable carrier. With respect to pharmaceutical compositions, the carrier can be any of those conventionally used and is limited only by chemico physical considerations, such as solubility and lack of reactivity with the active compound(s), and by the route of administration. The pharmaceutically acceptable carriers described herein, for example, vehicles, adjuvants, excipients, and diluents, are well known to those skilled in the art and are readily available to the public. It is preferred that the pharmaceutically acceptable carrier be one which is chemically inert to the active agent(s) and one which has no detrimental side effects or toxicity under the conditions of use. In some embodiments, the pharmaceutical compositions of the present disclosure further include at least one additional biologically active agent (e.g., an antibiotic agent or the like). In some embodiments, the pharmaceutical compositions of the present disclosure lack a pharmaceutically acceptable carrier.

The choice of carrier will be determined in part by the chemical properties of the vaccines as well as by the particular method used to administer the vaccines. Accordingly, there are a variety of suitable formulations of the pharmaceutical composition of the invention. The following formulations for intranasal, parenteral, subcutaneous, intravenous, intramuscular, intradermal, intraarterial, intrathecal and intraperitoneal administration are exemplary and are in no way limiting. More than one route can be used to administer the first and second vaccine, and in certain instances, a particular route can provide an immediate and more effective response than another route.

Injectable formulations are in accordance with the present disclosure according to some embodiments. Formulations for effective pharmaceutical carriers for injectable compositions are well-known to persons having ordinary skill in the art (see, e.g., Pharmaceutics and Pharmacy Practice, J.B. Lippincott Company, Philadelphia, PA, Banker and Chalmers, eds., pages 238 250 (1982), and ASHP Handbook on Injectable Drugs, Trissel, 14th ed., (2007)).

In accordance with some embodiments, the vaccines of the present invention can be administered other ways known in the art. For example, the vaccines can be administered via use of electroporation techniques. Suitable electroporation techniques are disclosed in U.S. Pat. Nos. 6,010,613, 6,603,998, and 6,713,291, all of which are incorporated herein by reference. Other physical approaches can include needle-free injection systems (NFIS) (e.g., as disclosed in U.S. Pat. No. 9,333,300, which is incorporated herein by reference), gene gun, biojector, ultrasound, and hydrodynamic delivery, all of which employ a physical force that permeates the cell membrane and facilitates intracellular gene transfer. Chemical vaccination approaches typically use synthetic or naturally occurring compounds (e.g., cationic lipids, cationic polymers, lipid-polymer hybrid systems) as carriers to deliver the nucleic acid into the cells.

In some aspects the vaccines disclosed herein are formulated in a lipid nanoparticle (LNP). The use of LNPs enables the effective delivery of chemically vaccines. Both modified and unmodified LNP formulated vaccines are optionally utilized. In some embodiments the vaccines disclosed herein are superior to conventional vaccines by a factor of at least 10 fold, 20 fold, 40 fold, 50 fold, 100 fold, 500 fold or 1,000 fold.

In one set of embodiments, lipid nanoparticles (LNPs) are provided. In one embodiment, a lipid nanoparticle comprises lipids including an ionizable lipid (such as an ionizable cationic lipid), a structural lipid, a phospholipid, and the RIC vaccine. Each of the LNPs described herein or otherwise known to persons having ordinary skill in the art may be used as a formulation for the vaccines described herein. In some embodiments, the LNP comprises an ionizable lipid, a PEG-modified lipid, a phospholipid and a structural lipid. In some embodiments, the LNP has a molar ratio of about 20-60% ionizable lipid: about 5-25% phospholipid: about 25-55% structural lipid; and about 0.5-15% PEG-modified lipid. In some embodiments, the LNP comprises a molar ratio of about 50% ionizable lipid, about 1.5% PEG-modified lipid, about 38.5% structural lipid and about 10% phospholipid. In some embodiments, the LNP comprises a molar ratio of about 55% ionizable lipid, about 2.5% PEG lipid, about 32.5% structural lipid and about 10% phospholipid. In some embodiments, the ionizable lipid is an ionizable amino or cationic lipid and the phospholipid is a neutral lipid, and the structural lipid is a cholesterol. In some embodiments, the LNP has a molar ratio of 50:38.5:10:1.5 of ionizable lipid: cholesterol:DSPC:PEG2000-DMG. Additional details regarding LNPs and other carriers that are optionally adapted for use with the vaccines of the present disclosure are also described in, for example, U.S. Patent Application Publication No. US 20200254086, which is incorporated by reference in its entirety.

For purposes of the present disclosure, the amount or dose of the vaccine administered should be sufficient to effect, e.g., a therapeutic or prophylactic response, in the subject over a selected time frame. The dose will typically be determined by the efficacy of the first and second vaccine and the condition of the given subject, as well as the body weight of that subject to be treated.

Typically, the attending physician will decide the dosage of first and second vaccine with which to treat each individual patient, taking into consideration a variety of factors, such as age, body weight, general health, diet, sex, to be administered, route of administration, and the severity of the condition being treated. By way of example and not intending to limit the invention, the dose of the vaccine is about 1 to 10,000 μg of vaccine to the subject being treated. In some embodiments, the dosage range of the vaccine is about 500 μg-6,000 μg of vaccine. In one exemplary embodiment, the dosage of the vaccine is about 3,000 μg.

### Example 1: A Self-Binding Immune Complex Vaccine Elicits Strong Neutralizing Responses Against Herpes Simplex Virus in Mice

### 1. Introduction

Antibodies are one of the most widely produced therapeutic agents, comprising the largest share of the global biopharmaceutical market. In 2021, the one-hundredth antibody therapy was approved by the FDA. While antibodies by themselves are highly useful, it is becoming increasingly common to fuse antibodies to other proteins of interest to imbue them with desirable properties. Fusion to IgG antibody often provides enhanced solubility and stability of the fusion partner due to the inherent stability of IgG molecules and allows simple and highly efficient purification via protein A/G affinity chromatography. Additionally, IgG fusions may have extended serum half-life, as IgG are protected from degradation in endosomes due to their ability to bind neonatal Fc receptor (FcRn).

Though less explored, IgG fusion molecules also have additional properties uniquely suited to the creation of potent vaccines. Antibody-antigen complexes are directly taken up by antigen-presenting cells such as dendritic cells, macrophages, and B cells via the interactions of the IgG Fc with FcRn receptors, complement receptors and Fcγ receptors. However, not all antibody-antigen molecules are potent immunogens. When repetitive antigens are bound by antibody, they form larger immune complexes (ICs) which are more potent activators of immune receptors than monomeric antibody-bound antigen. For instance, the complement receptor C1q requires simultaneous engagement of its six head regions with six IgG Fc regions, and thus monomeric antibody poorly activates complement, whereas multimeric ICs potently activate complement. Complement activation leads to iC3b coating of the ICs as well as release of complement anaphylatoxins, resulting in the recruitment of immune cells to the site of vaccination, deposition of complexed antigen onto follicular dendritic cells, and subsequent stimulation of both B cell and T cell immunity. In a similar fashion, larger ICs, but not monomeric antibody-bound antigen, can efficiently cross-link low affinity Fcγ receptors, leading to further enhanced uptake and stimulation by antigen presenting cells.

To harness the benefits of IgG fusions, several vaccine platforms have been designed. Perhaps the simplest method is to simply fuse an antigen of interest to the IgG Fc. The most successful example of this strategy is a SARS-CoV-2 vaccine consisting of interferon-α, the pan HLA-DR-binding epitope (PADRE), and the SARS-CoV-2 spike receptor binding domain fused to IgG1 Fc. Compared to immunization with the receptor binding domain alone, the Fc fusion was found in higher abundance in lymph nodes, and safely generated strong Th1, Th2, CD8+ and neutralizing antibody responses in rhesus macaques and in humans. Intriguingly, a vaccine comprising herpes simplex virus (HSV) 2 glycoprotein D (gD) fused to an IgG Fc could be efficiently administered mucosally, as FcRn receptors mediate uptake of IgG across mucosal epithelial surfaces. In a different strategy, mixing specific antibodies with antigens to form an immune complex (IC) has been used to focus immune responses towards favorable antigenic sites on tick-borne encephalitis virus and HIV. Vaccination with sialylated ICs targeting influenza hemagglutinin was found to improve the breadth and potency of anti-influenza antibodies by selecting for high affinity B cells. These studies underscore the unique benefits of IgG fusion vaccines.

While some Fc fusions may spontaneously form multimeric structures capable of engaging complement and low affinity Fcγ immune receptors, their stability and antigenicity appear to be strongly dependent on the characteristics of the fusion partner, such as whether the fused antigen forms multimers. Therefore, strategies have been developed to generate consistently immunogenic antigen-antibody structures. In one strategy, antigen was delivered on an IgG1 containing the IgM tailpiece and coexpressed with the J chain, forming pentameric and hexameric molecules. These constructs efficiently engaged C1q and low affinity Fcγ receptors, providing robust B cell and T cell immunity in mice and in human adenotonsillar tissue against the dengue virus envelope protein. This strategy was also successful when used to orally deliver an antigen derived from porcine epidemic diarrhea virus.

We designed a vaccine platform consisting of self-multimerizing ICs capable of forming highly immunogenic clusters with antigens of interest, called recombinant immune complexes (RICs). In this system, the antigen is fused to the well-characterized mAb 6D8 which has been tagged with its own binding site, allowing multiple antigen-antibody molecules to bind to each other to form larger complexes. In the present example, to investigate the key differences between traditional IC and the RIC system, we compared the immunogenic properties of herpes simplex virus 2 (HSV-2) glycoprotein D (gD) delivered either via traditional monomeric IC or via self-interacting multimeric RIC.

### 2. Results

2.1 Production and Characterization gD-IC and gD-RIC

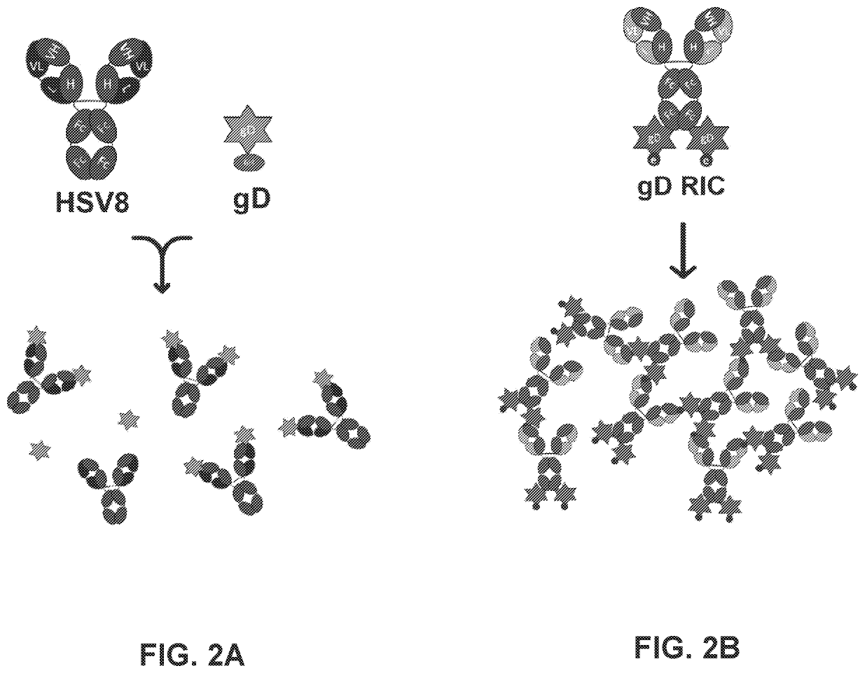

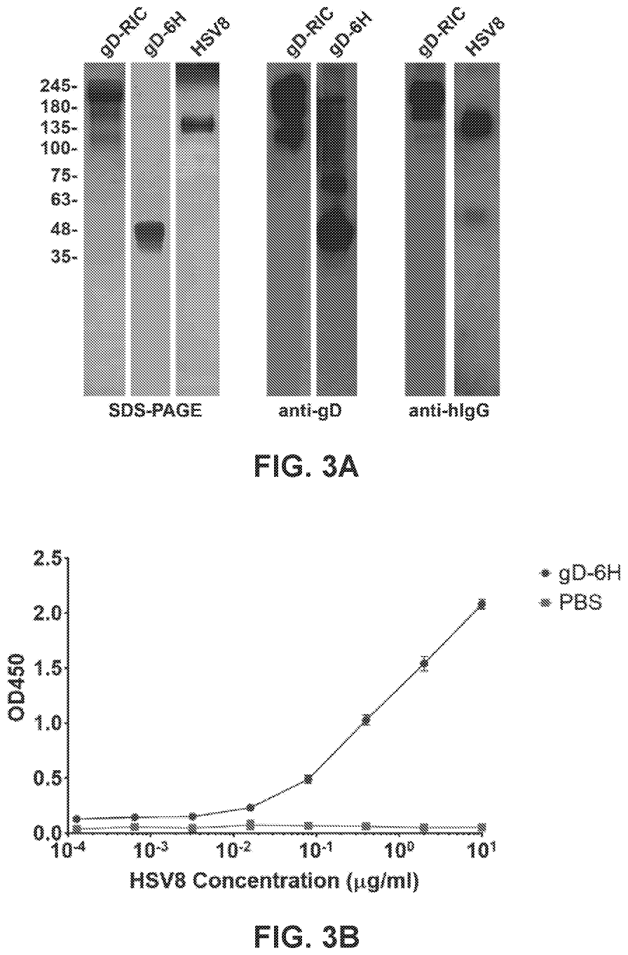

To create a traditional IC targeting HSV, the neutralizing mAb HSV8, which recognizes a conformational epitope in gD, was expressed in plants and purified along with the soluble ectodomain from HSV-2 gD (amino acids 26-331) containing a 6-histidine tag (FIG. 2, constructs “HSV8” and “gD”). By SDS-PAGE, HSV8 formed bands at ˜150 kDa, while gD formed bands around ˜48 kDa, which is expected for glycosylated gD (FIG. 3A). HSV8 readily detected gD via nonreducing western blot (FIG. 3A) but not by reducing western blot (data not shown). To further verify the binding of plant-made HSV8 and gD in solution, an ELISA was performed. HSV8 showed robust binding to gD at concentrations as low as 3 nM (FIG. 2B), indicating that IC formation occurs readily in solution.

Next, an RIC vector targeting HSV was created by inserting the DNA sequence encoding HSV-2 gD (amino acids 26-331) at the C-terminus of the heavy chain of the human IgG1 6D8 tagged with its own binding site (FIG. 2, construct “gD-RIC”). When purified gD-RIC was probed with HSV8 or anti-human IgG by western blot, a band was seen around the expected size of ˜218 kDa for fully formed gD-RIC containing both human IgG1 and gD antigen (FIG. 3).

2.2 gD-RIC Form Larger Complexes with Improved Immune Receptor Binding Compared to gD-IC

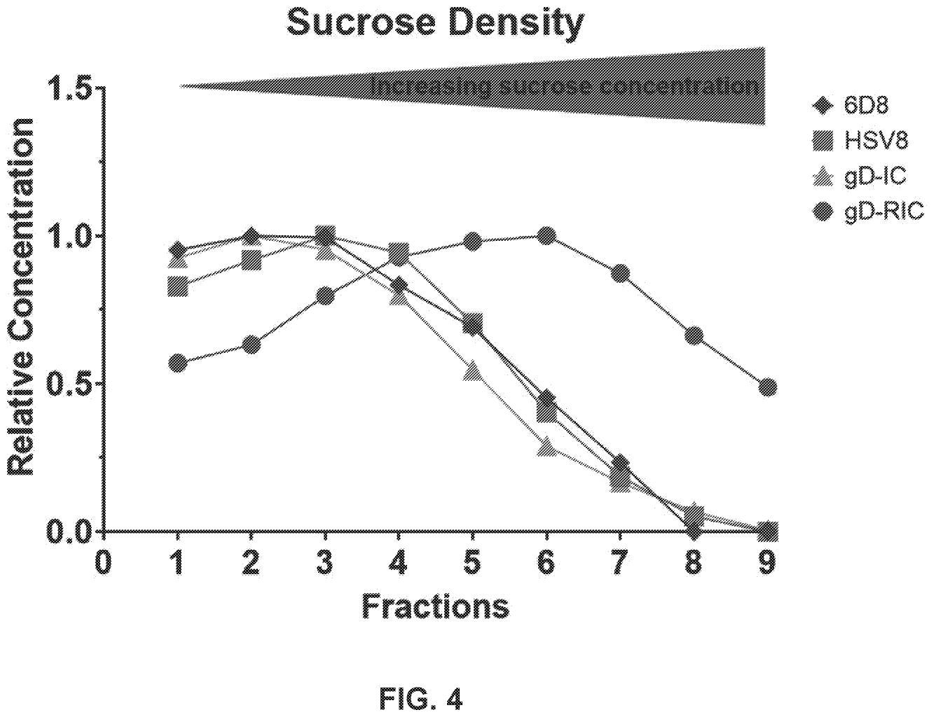

To determine whether gD-RIC form larger complexes than gD-IC, both constructs were analyzed by sucrose gradient sedimentation using the monomeric antibodies 6D8 and HSV8 as controls. Since gD-RIC contains potentially two gD molecules per antibody molecule, gD-IC were also prepared by preincubating HSV8 with gD at a molar ratio of 1:2. Whereas gD-IC did not display notable differences in density compared to the controls, gD-RIC was found to sediment substantially faster, forming a broad peak consistent with the formation of large heterogenous complexes (FIG. 4).

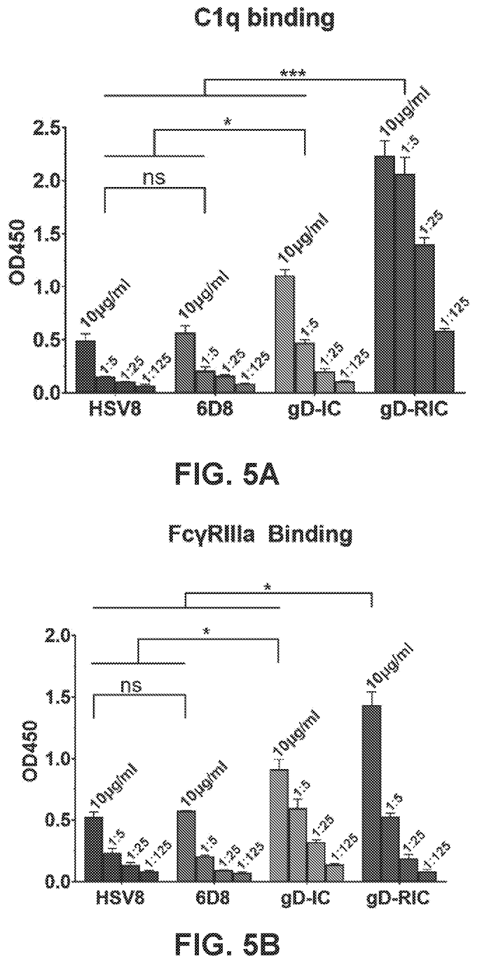

Complement receptor C1q preferentially binds multimeric IgG, with hexamer or larger complexes having the strongest binding. All antibody constructs were expressed in plants silenced for the plant-specific glycans fucose and xylose, which has been shown to improve antibody immune receptor binding. The mAbs 6D8 and HSV8 showed minimal binding to C1q, while gD-HSV8 IC showed somewhat improved binding (FIG. 5A, p<0.05). By stark contrast, gD-RIC showed a 25-fold increase in C1q binding compared to gD-IC (FIG. 5A, p<0.001, compare gD-RIC column 3, representing 25-fold dilution, to gD-IC column 1, undiluted). Low affinity receptor FcγRIIIa binding was also measured by ELISA. Both IC and RIC (FIG. 5B, p<0.05) displayed improved FcγRIIIa binding compared to monomeric controls, with RIC showing the highest binding (FIG. 5B, p<0.05).

2.3 gD-RIC are Highly Immunogenic in Mice Compared to gD-IC

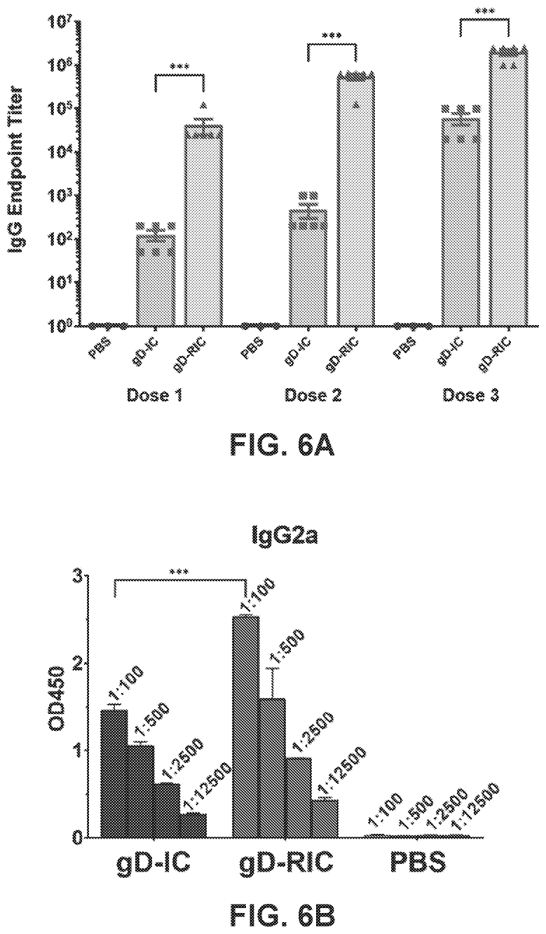

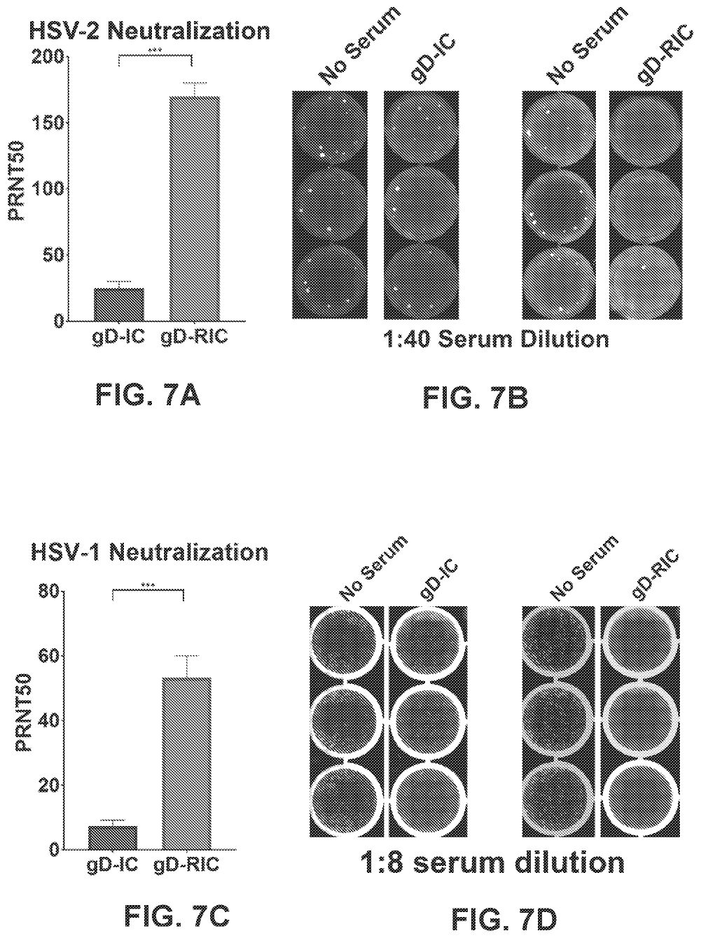

To test the in vivo immunogenicity of each construct, BALB/c mice were immunized three times with 4 μg gD delivered either as gD-IC or gD-RIC without adjuvant. After each dose, the resulting mouse serum was analyzed for gD-specific antibody titers by ELISA. Strikingly, mice immunized with gD-RIC produced titers that exceeded those of the IC-immunized mice by 332-fold, 1162-fold, and 33-fold after doses 1, 2 and 3 respectively (FIG. 6A, p<0.001). gD-RIC produced 5-fold higher levels of IgG2a antibodies compared to gD-IC (FIG. 6B, p<0.05). Finally, the neutralization of herpes simplex virus 2 (HSV-2) using serum from gD-IC or gD-RIC immunized mice was compared. gD-RIC serum neutralized HSV-2 significantly more than gD-IC serum (FIG. 7A-B, p<0.001). To assess the cross-neutralizing potential of the immune serum, neutralization with HSV-1 was also performed. gD-RIC serum cross-neutralized HSV-1 significantly more than gD-IC serum (FIG. 7C-D, p<0.001).

### 3. Discussion

HSV infection of neonates has as high as a 50% chance of developing disseminated disease or encephalitis, with current drug options still leaving approximately 70% of neonates with long-term neurological sequelae. Neonatal infection often occurs (55%) if the mother becomes infected for the first time during pregnancy, whereas there is minimal risk if the mother has previously been infected (<1%), likely due to the transfer of maternal antibodies. Building on these findings, it has recently been shown that vaccination of the mother can also prevent neonatal HSV infection in a mouse model. These results underscore the need for safe, effective, and cheap HSV vaccines. In the present study, we show that a self-binding antibody complex formed with gD (gD-RIC) induces robust gD-specific antibody production and neutralizes both HSV-1 and HSV-2, strongly outperforming a traditional IC composed of gD simply mixed with a neutralizing antibody (gD-IC) (FIG. 5, FIG. 6).

Past research has repeatedly demonstrated that the delivery of IC composed of an antigen mixed with antisera can enhance the immune response towards a given antigen compared to antigen delivery alone. However, many studies have found inconsistent results, including reduced immunogenicity of IC vaccines and, despite being studied for over half a century, IC-based therapeutics have failed to produce a single FDA-approved vaccine. In this example, both vaccine preparations contain equivalent total amounts of both antibody and antigen delivered in the same ratio: approximately one antibody molecule per two gD molecules. Nevertheless, gD-RIC produced strikingly higher immune responses, up to 1,000-fold higher antigen-specific antibody titers after 2 doses (FIG. 6A). The immunogenicity of a given IC depends strongly on the individual properties of each antibody and the characteristics of its cognate antigen. For instance, an IC formed by repetitive antigens can spontaneously form larger immune complexes, whereas IC composed of monomeric antigen and monoclonal antibodies cannot form larger complexes (FIG. 2A). Several important immune receptors have evolved to preferentially activate in the presence of highly complexed antibody bound to repetitive pathogen epitopes, including complement receptor C1q, which initiates the complement cascade, and the low affinity receptor FcγRIIIa, considered to be one of the main effector FcγRs on immune cells. Monomeric antigen-antibody complexes have reduced capacity to induce strong immune responses because they cannot activate these pathways. An IgG fusion vaccine targeting dengue virus envelope protein domain Ill produced stronger B cell and T cell responses when delivered in a polymeric form compared to a monomeric form in human adenotonsillar tissue. Consistent with this prior work, our data shows that gD-RIC forms large complexes with strongly improved ability to bind C1q and greater overall antigenicity (FIGS. 4-7). By contrast, gD-IC forms smaller complexes with severely impaired immune receptor binding and antigenicity (FIGS. 4-7). An optimal vaccine must be cost-effective, eliciting strong responses with as few doses as possible. Only after the third dose did the IC group achieve a mean titer equivalent to a single dose of RIC (FIG. 6A), highlighting the ability of gD-RIC to produce very strong immune responses with minimal doses without adjuvant. We have previously found that an Ebola RIC produced large heterogenous complexes, though with somewhat reduced C1q binding and immunogenicity compared to the present study due to incorporation of endogenous plant glycans (data to be presented elsewhere). Oligomeric antigen-antibody vaccine preparations such as RIC have now been shown to strongly enhance antigen immunogenicity using a variety of antigens, including tetantus toxin, Ebola glycoprotein 1, dengue virus envelope protein, human papillomavirus L2, Zika virus envelope, and a short 23 amino acid peptide comprising the ectodomain of influenza matrix 2 protein, underscoring their broad potential to consistently and efficiently induce strong immune responses to a variety of large and small antigens.

It could be argued that the potent immunogenicity observed by gD-RIC is due in part to a mouse immune response directed against human IgG1. Notably, gD-IC and gD-RIC contain identical amounts of human IgG1. We observed modest levels of antibodies targeting the IgG1 backbone in gD-RIC, though no evidence of strong titers generated against the 6D8 epitope tag itself (FIG. 11). To determine whether gD-RIC retains its strong immunogenicity without the human antibody backbone, we generated gD-RIC utilizing a mouse IgG2a backbone and found no statistical differences in antigen-specific antibody titers or virus neutralization compared to human gD-RIC constructs. Other groups have shown that antibody complexes utilizing a mouse IgG backbone were still potent enhancers of antigen immunogenicity in mice, consistent with our observations. Indeed, that larger immune complexes make potent immunogens is well-established, as this feature is undesirable for intravenous antibody therapy. It has been known for over half a century that antibody aggregates can cause pathology such as serum sickness, and immune complex deposition is a driver of inflammation in diseases such as lupus erythematosus. Hypersensitivity reactions caused by immune complexes are a concern with all antibody therapies. Therefore, these concerns must be addressed to ensure the safety of immune complex vaccines. Notably, antibody therapies are commonly administered with doses in the range of several grams of antibody. On the other hand, typical intramuscular injection of a vaccine preparation delivers 1,000-50,000 times lower doses, in the microgram range. During natural infection, immune complex formation is a necessary and desirable consequence of an effective immune response, as antibodies directed against repeated antigens, such as viral capsids, will inevitably form larger heterogeneous antibody-antigen complexes. Importantly, the immunostimulatory nature of repetitive antigens, which would generate large immune complexes upon repeated vaccination, has resulted in the success of safe and effective virus-like particle vaccines such as Gardasil. The RIC platform allows this enhanced immunogenicity to be conferred to antigens that otherwise do not self-assemble into larger complexes. We have not observed any toxic effects in mice across multiple RIC vaccine studies.

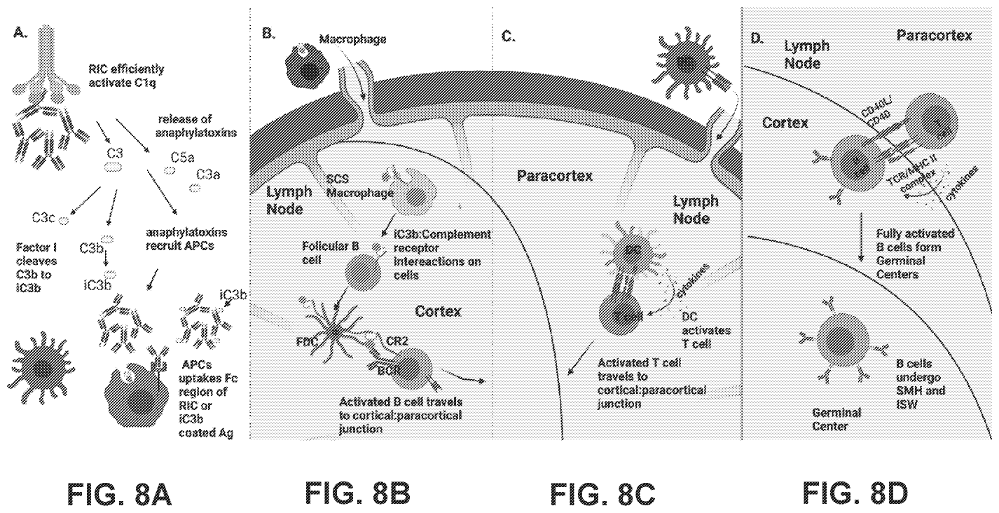

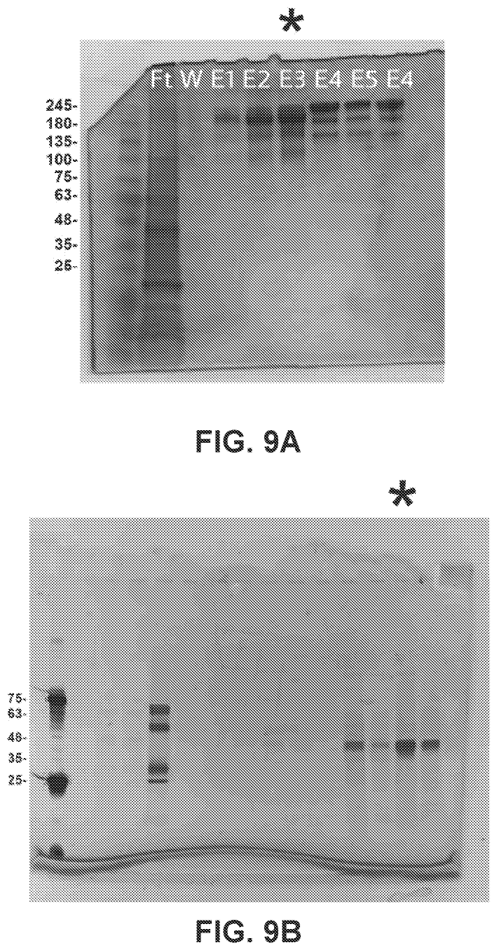

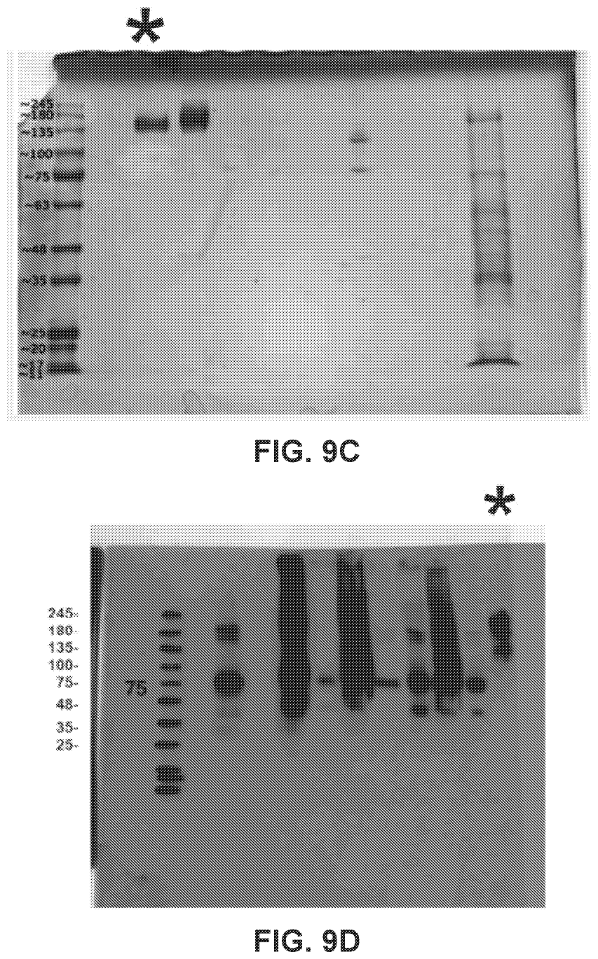

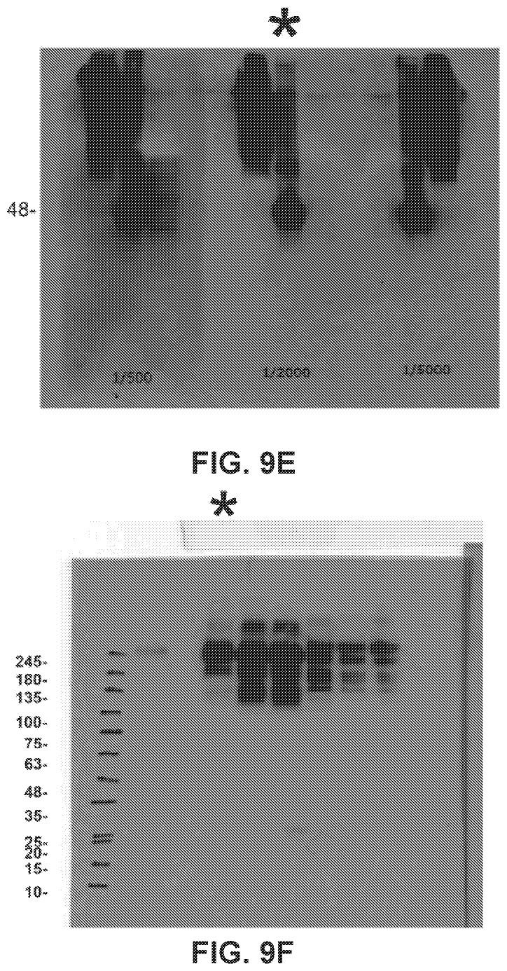

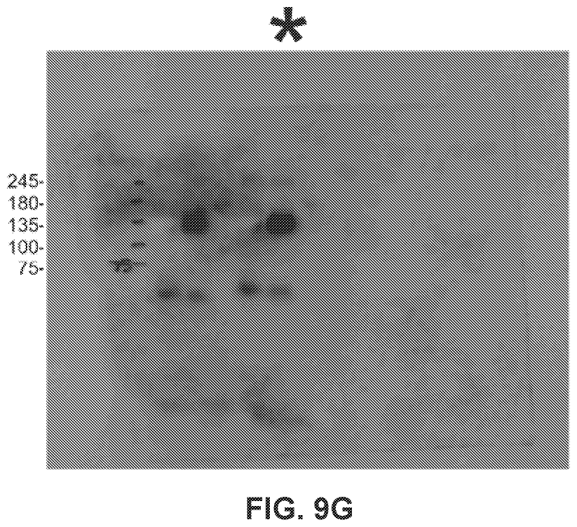

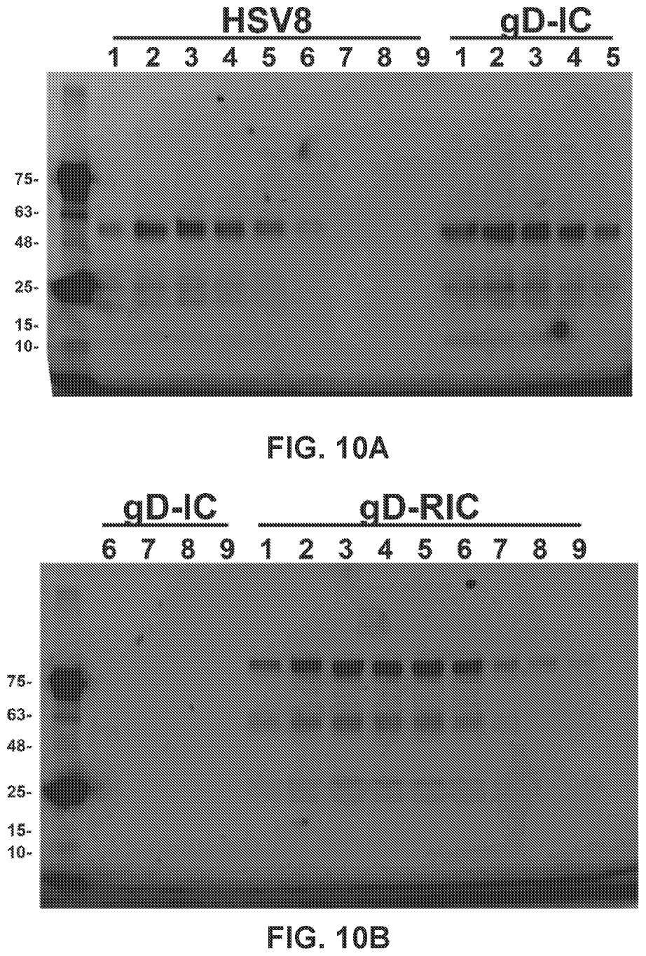

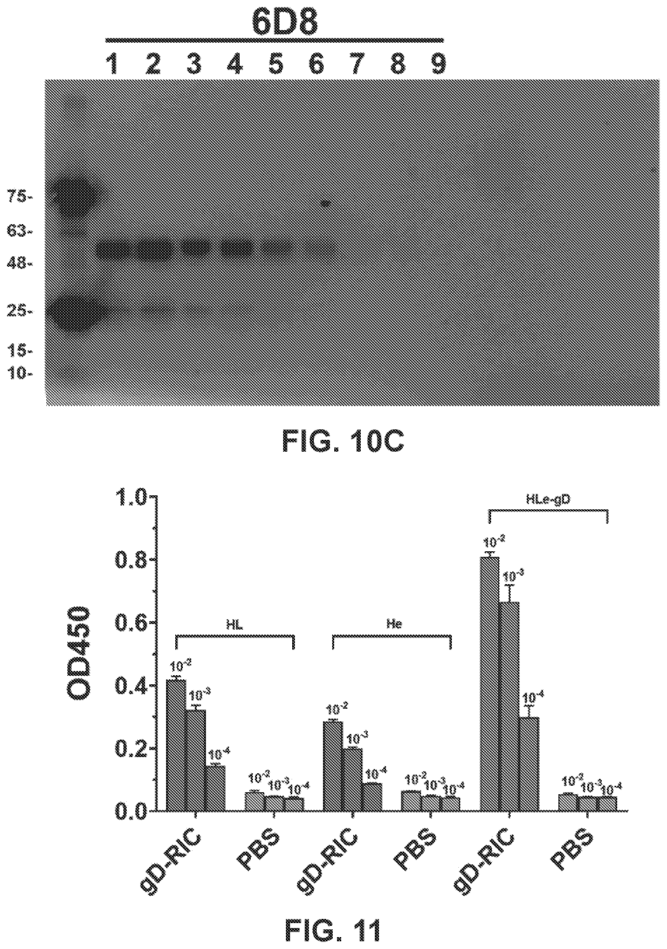

We propose several mechanisms by which the RIC platform may enhance antigen immunogenicity, outlined in FIG. 8. Multivalent ICs have been shown to be potent activators of complement (FIG. 5A). Although the complement system is canonically considered innate, in recent decades, the role of compliment in activating the adaptive immune system has been illustrated in several ways. Primarily, aggregated antibody complexes, like RICs, activate the complement cascade, which results in the cleavage of complement proteins, including C5 and C3, releasing C3a and C5a as potent anaphylatoxins. These anaphylatoxins cause mast cell and basophil degranulation, leading to a release of vasoactive substances which increase blood flow to the site of vaccination. Multiple cell types are then activated through anaphylatoxin binding to G-protein coupled receptors on phagocytic cells that can serve as APCs, including dendritic cells and macrophages. Activation of these APCs causes them to travel to the lymph node, leading to activation of B and T cells; it can also result in pro-inflammatory cytokine release, which successively recruits more immune cells to the vaccination site. Increases in vascular permeability in these areas can also encourage the movement of antigen-bearing APCs to draining lymph nodes, encouraging efficient elicitation of adaptive immune responses (FIG. 8A).

Additionally, complement activation results in the production of iC3b, which, upon ligation with the CR2 receptor on follicular dendritic cells, is necessary for full B cell activation in the lymph node. In fact, a decline in circulating levels of complement can result in impaired antigen-specific antibody responses, indicating the requirement of complement presence for effective humoral responses to pathogens (FIG. 8B). Noting that RICs elicit high levels of antigen-specific antibodies and that CD4 T cell help in the form of cytokine and costimulatory signals is required for isotype switching and affinity maturation of antibodies to occur, we further speculate that RICs play a role in an augmented presentation of antigen to T cells and subsequent T cell activation. Complement proteins have been shown more recently to play a role in activating and regulating both CD4+ and CD8+ T cell responses. As T cells are initially activated by DCs that have traveled to the lymph node, and DCs can be recruited and activated with the help of anaphylatoxins at the vaccination site, this potential T cell regulation could occur via the increased presentation of Ag by DCs in the lymph node to T cells. Additionally, since RICs contain numerous Ig domains, FcγR on DCs can more readily take up RICs linked to antigen, allowing for enhanced internalization of antigen and increased presentation of antigen to T cells in the lymph nodes. Further, there is evidence that complement activation can elicit effective T cell signaling allowing for proper T cell activation (FIG. 7C). After full activation of T and B cells, both will migrate to the cortical: paracortical junction, where CD40:CD40L interactions will occur between T and B cells. This interaction not only allows for full T cell activation but also allows B cells to gain T cell help via cytokines and costimulatory molecules, necessary for them to create germinal centers and undergo somatic hypermutation and isotype switching, generating the Ag-specific responses we have illustrated following vaccination with RIC (FIG. 8D). Future studies elucidating the precise mechanisms underlying the observed immunogenicity of RICs could lead to additional enhanced vaccination strategies and could be extended to other vaccine platforms.

The induction of neutralizing antibody titers has long been a gold standard for vaccine research, though in recent years it has been more widely appreciated that non-neutralizing antibodies are also important for protection against most viruses, including HSV. Compared to gD-IC, gD-RIC elicited higher total IgG titers (FIG. 6A), higher HSV-2 and HSV-1 specific neutralization titers (FIG. 7), and higher levels of IgG2a antibodies (FIG. 6B). While BALB/c mice mainly produce the IgG1 antibody subclass, the antibody subclass IgG2a is an indicator of a stronger Th1 type response and these antibodies have important Fc-mediated antiviral functions. Due to serum limitations, neutralization studies were performed with dose 3 serum, where differences between gD-IC and gD-RIC were less pronounced than after doses 1 or 2 (FIG. 6A). The differences between gD-IC and gD-RIC are likely reduced after dose 3 because there are diminishing returns from repeated vaccination, with the gD-RIC antibody responses in the serum eventually reaching maximal levels.

Another rationale for the design of RIC vaccines is the lack of antibody binding to the target antigen itself. Instead, RIC form by binding a defined epitope tag separated from the antigen via linker (FIG. 2B), allowing the same universal RIC platform to function with any desired antigen. By contrast, a traditional IC preparation requires a new antibody to be identified and tested empirically for each new vaccine antigen. Direct antigen binding in IC may reduce the interaction of B cells specific for the already bound epitope, a phenomenon known as epitope masking. A malaria vaccine candidate was found to have limited B cell expansion after administering a third dose due to epitope masking, but increased diversification of humoral responses. An HIV IC vaccine candidate retained similar overall antigen titers compared to vaccination with antigen alone, however epitope masking by the IC focused immune responses towards more desirable epitopes. Overall, epitope masking can be advantageous or disadvantageous depending on context. In the case of gD-IC, the soluble ectodomain of HSV-2 gD used in this study comprises 306 amino acids, spanning 17 known major epitope regions which have been extensively studied. HSV8 binds an epitope located between amino acids 234-270. Serum from immunization with HSV-2 gD was found to contain antibodies targeting a variety of linear and conformational epitopes throughout gD. As numerous anti-gD antibodies were able to bind even when epitopes which bind the same region as HSV8 were blocked, it is unlikely that epitope masking played a large role in the overall differences gD-specific titers observed between gD-IC and gD-RIC. However, after dose 3, we observed only an ˜8-fold increase in mean neutralization titers with gD-RIC, despite a ˜20-fold increase in mean gD-specific antibody titers (FIGS. 6-7). While these differences would be insignificant compared to the very large differences in total antibody titers after dose 2 (FIG. 6), we cannot exclude the possibility that epitope masking present in gD-IC may have modestly focused antibody production towards more neutralizing epitopes compared to gD-RIC. Alternatively, gD fusion to the IgG1 heavy chain, the epitope tag fusion itself, or other factors, may have inhibited proper formation of some neutralizing epitopes in gD-RIC. These findings suggest potential room for future optimization of the RIC platform. By modifying the strength of epitope tag binding, RIC variants have been produced which were smaller, more homogenous, and more soluble, while still maintaining strong immunogenicity. Future studies are needed to investigate additional possible optimizations to the RIC platform, such separating the antigen with longer linkers, employing different self-multimerization strategies, or by utilizing direct antigen binding to confer advantageous epitope masking.

### 4. Experimental Procedures

### 4.1 Vector Construction

The construction details of pBYKEMd2-HSV8 have been previously published. A construct containing the HSV gD antigen fused to a 6H tag was created by digesting pBYe3R2K2Mc-BAZsE6H with XhoI-SpeI to produce the expression vector fragment. The insert with the gD coding sequence was derived from a PCR-amplification of the cloned gene in pCRblunt-gD. The final construct was named pBYe3R2K2Mc-gD306-6H (sometimes referred to as “gD”).

A RIC vector containing gD linked to the humanized, 6D8 antibody C-terminus was created by PCR-amplifying pCRblunt-gD with end-tailoring primers, gD-Bam-F (5′-GGGGATCCAAATATGCATTAGCTGATCCTAGTC-3′ (SEQ ID NO: 15)) and gD306-Spe-R (5′-GCAACTAGTATGGTGTGGAGCAACATC-3′ (SEQ ID NO: 16)), to add BamHI-SpeI restriction sites. The PCR product was digested BamHI-SpeI and ligated with the vector derived from pBYR11eM-h6D8ZE3 to produce pBYR11eM-h6D8gD (“gD-RIC”).

4.2 Agroinfiltration of Nicotiana benthamiana Leaves

Binary vectors were separately introduced into Agrobacterium tumefaciens EHA105 by electroporation. The resulting strains were verified by restriction digestion or PCR, grown overnight at 30° C., and used to infiltrate leaves of 5- to 6-week-old N. benthamiana maintained at 23-25° C. Briefly, the bacteria were pelleted by centrifugation for 5 min at 5,000 g and then resuspended in infiltration buffer (10 mM 2-(N-morpholino)ethanesulfonic acid (MES), pH 5.5 and 10 mM MgSO4) to OD600=0.2, unless otherwise described. The resulting bacterial suspensions were injected by using a syringe without needle into leaves through a small puncture. It has been previously shown that IgG-based vaccines have enhanced immune receptor binding properties when produced in glycan-modified plants, therefore transgenic plants silenced for xylosyltransferase and fucosyltransferase were employed. Plant tissue was harvested at 5 days post infiltration (DPI).

### 4.3 Protein Extraction, Expression and Purification

Constructs gD-RIC, HSV8, and 6D8 were purified by protein G affinity chromatography. Agroinfiltrated leaves were blended with 1:3 (w:v) ice cold extraction buffer (25 mM Tris-HCl, pH 8.0, 125 mM NaCl, 3 mM EDTA, 0.1% Triton X-100, 10 mg/mL sodium ascorbate, 0.3 mg/mL phenylmethylsulfonyl fluoride), stirred for 30 min at 4° C., and filtered through miracloth. To precipitate endogenous plant proteins, the pH was lowered to 4.5 with 1M phosphoric acid for 5 min while stirring on ice, then raised to 7.6 with 2M Tris base. Following centrifugation for 20 min at 16,000 g, the clarified extract was loaded onto a Protein G column (Thermo Fisher Scientific, Waltham, MA, USA) following the manufacturer's instructions. Purified proteins were eluted with 100 mM glycine, pH 2.5, directly into collection tubes containing 1M Tris-HCl pH 8.0 to neutralize the elution buffer and stored at −80° C. Purified protein concentration was measured by A280 absorbance, ELISA, and gel quantification.

gD-His expressed from pBYe3R2K2Mc-gD6H was purified by metal affinity chromatography. Protein was extracted as described above, but without acid precipitation. The clarified extract was loaded onto a column containing TALON Metal Affinity Resin (BD Clontech, Mountain View, CA) according to the manufacturer's instructions. The column was washed with PBS and eluted with elution buffer (PBS, 150 mM imidazole, pH 7.4). Peak protein elutions were identified by SDS-PAGE, pooled, dialyzed against PBS, and stored at −80° C. Protein concentration was measured by A280 absorbance and gel quantification.

### 4.4 SDS-PAGE and Western Blot

Plant protein extracts or purified protein samples were mixed with SDS sample buffer (50 mM Tris-HCl, pH 6.8, 2% SDS, 10% glycerol, 0.02% bromophenol blue) and separated on 4-15% stain-free polyacrylamide gels (Bio-Rad, Hercules, CA, USA). For reducing conditions, 0.5M DTT was added, and the samples were boiled for 10 min prior to loading. Polyacrylamide gels were visualized and imaged under UV light, then transferred to a PVDF membrane. The protein transferred membranes were blocked with 5% dry milk in PBST (PBS with 0.05% tween-20) overnight at 4° C. and probed with goat anti-human IgG-HRP (Sigma-Aldrich, St. Louis, MO, USA diluted 1:5000 in 1% PBSTM) for IgG detection; or, probed with human HSV8 (plant-made, diluted 1:1000 from 1 mg/ml in 1% PBSTM) for gD-6H detection; or, probed with the mouse anti-gD mAb H170 (Santa Cruz Biotechnology, TX, USA, diluted 1:1000 in 1% PBSTM) for gD-RIC detection. Bound antibody was then detected with either anti-human IgG-HRP (Sigma-Aldrich, St. Louis, MO, USA, diluted 1:5000 in 1% PBSTM) for HSV8, or with anti-mouse IgG-HRP (Southern Biotech, AL, USA, diluted 1:5000 in 1% PBSTM) for H170. Bound antibodies were detected with ECL reagent (Amersham, Little Chalfont, United Kingdom).

### 4.5 ELISA

IC were prepared by incubating gD-6H and HSV8 at a 2:1 molar ratio to mimic the ratio of antigen and antibody present in gD-RIC for 2 hours at room temperature. During this time, purified RIC aliquots were thawed, and RIC and IC were serially diluted in 1% PBSTM for ELISA. RIC and IC were both set to a starting concentration of 10 μg/ml (including antigen and antibody weight. For immune receptor binding, 96-well medium-binding polystyrene plates (Thermo Fisher Scientific, Waltham, MA, USA) were coated with 15 μg/ml human complement C1q or human FcγRIIIa (PFA, MilliporeSigma, MA) in PBS for 1.5 hours at 37° C. For IC binding experiments, the plates were instead coated with 15 μg/ml plant-made gD-6H. The plates were washed 3 times with PBST, and then blocked with 5% dry milk in PBST for 30 minutes. After washing 3 times with PBST, for immune receptor binding experiments, purified IC or RIC were added at 10 μg/ml with a 5-fold serial dilution and were incubated for 1.5 hours at 37° C. For IC binding, HSV8 was added at initial concentration of 10 μg/ml with 5-fold serial dilutions. After washing 3 times with PBST, bound IgG was detected by incubating with a 1:500 dilution of an anti-human IgG (whole molecule) HRP-labeled probe (Sigma-Aldrich, St. Louis, MO, USA) for 1 hour at 37° C. The plates were washed 4 times with PBST, developed with TMB substrate (Thermo Fisher Scientific, Waltham, MA, USA), stopped with 1M HCl, and the absorbance was read at 450 nm.

### 4.6 Sucrose Gradient Density Centrifugation

Purified 0.5 mg samples (300 μl) of IC, RIC, mAbs, or PBS control were loaded onto discontinuous sucrose gradients consisting of 300 μl layers of 5, 10, 15, 20, and 25% sucrose in PBS in 2.0 ml microcentrifuge tubes and centrifuged at 21,000 g for 24 h at 4° C. Nine fractions (200 μl) were collected, and the total protein content of each fraction was measured via spectrophotometry. The A280 absorbance of the PBS control fractions were subtracted from each corresponding fraction of IC or RIC. The highest absorbance value was arbitrarily assigned the value of “1” and the other fractions were calculated relative to this value. Representative results from 3 independent experiments are shown.

### 4.7 Immunization of Mice and Sample Collection

Groups (n=6) of female Balb/c mice, 6-7 weeks old, were immunized subcutaneously with gD-IC prepared with a 1:2 molar ratio of HSV8 to gD, or gD-RIC. An equivalent amount of 4 μg of gD was delivered per dose. The constructs were first analyzed by SDS-PAGE to detect any cleavage products, then quantified by the ImageJ software and spectroscopy to determine the percentage of gD-containing antigen. Three mice were immunized with PBS as a negative control. No adjuvant was used for any group. Doses were delivered on days 0, 28, and 56. Serum was collected by submandibular bleed as described (Santi et al., 2008) on days 0, 28, and 56, and 86. All animals were handled in accordance with the Animal Welfare Act and Arizona State University IACUC.

### 4.8 Antibody Measurements

Mouse antibody titers specific for gD, 6D8 variants, or gD-RIC were measured by ELISA. Purified gD, 6D8 variants, or gD-RIC (15 μg/ml) were bound to 96-well high-binding polystyrene plates by a 1-hour incubation at 37° C. (Corning Inc, Corning, NY, USA). The plates were then washed with PBST (PBS with 0.05% tween-20) and blocked with 5% nonfat dry milk in PBST. After the wells were washed with PBST, the diluted mouse sera (5-fold serial dilutions from 1:40 to 1:3, 125,000 for gD, or 10-fold serial dilutions starting from 1:100 for 6D8 variant and gD-RIC ELISA) from each bleed were added and the plate incubated at 37° C. for 1 hour. After washing with PBST, the mouse antibodies were detected by a 1-hour incubation with either a polyclonal goat anti-mouse IgG-horseradish peroxidase conjugate (Sigma-Aldrich, St. Louis, MO, USA) or a IgG2a horseradish peroxidase conjugate (Santa Cruz Biotechnology, Dallas, TX, USA). The plate was then developed with TMB substrate according to the manufacturer's instructions (Thermo Fisher Scientific, Waltham, MA, USA) and the absorbance was read at 450 nm. The endpoint titers were taken as the reciprocal of the lowest dilution which produced an OD450 reading twice the background. Statistical analysis between the vaccinated groups was carried out using one-way ANOVA with Tukey's post-test for multiple comparisons.

### 4.9 Cell Culture and Virus Propagation

Vero cells (African green monkey kidney cells, ATCC) were cultured in a 5% CO₂ incubator with Dulbecco's Modified Eagle's Media (DMEM, Cytiva), supplemented with 10% fetal bovine serum (FBS, Gibco) and 1% Penicillin-Streptomycin (Pen-Strep, Gibco). A recombinant HSV-2 strain expressing green fluorescent protein (GFP) was a kind gift from Orkide Koyuncu (UC Irvine). The HSV-1 OK14 strain, expressing a red fluorescent protein (RFP)-tagged capsid protein, was previously described (Song et al., 2016). Viral stocks were grown on Vero cells incubated with viral media (DMEM supplemented with 2% FBS and 1% Pen-Strep). Virus stocks were harvested once significant cytopathic effect was observed and stored at −80 C in 2% HEPES buffer (Gibco). Virus stocks were titered by serial dilution plaque assay, as described below.

### 4.10 Neutralization Assay and Plaque Imaging

24-well plates were seeded 2×10⁵ Vero cells per well and incubated overnight. Mouse serum from all six individual mice was combined to a final volume of 20 μL and then diluted to a 1:5 concentration in 100 μL of viral media. This initial 1:5 dilution was then serially diluted two-fold in 100 μL volumes for an additional five dilutions. HSV-2 and HSV-1 OK14 stocks were diluted to a concentration of 500 plaque-forming units (PFU)/mL, and 100 μL (50 PFU) of this diluted working stock was mixed with each serum dilution, and incubated for 1 hour at 37° C. Vero cells were then inoculated with the serum-virus solutions for 1 hour at 37° C. Next, the inoculum was aspirated off and the cell monolayer was overlaid with 1 mL of Methocel-thickened viral medium. At 48 hours post-infection, fluorescent foci were imaged using a Nikon Ti2-E inverted widefield fluorescence microscope in the ASU Biodesign Imaging Core Facility. This microscope is equipped with a SpectraX LED light source, providing 470/24 nm light for GFP excitation or 550/15 nm light for RFP excitation. Fluorescence emission was captured using a Photometrics Prime95B sCMOS camera. Nikon NIS Elements software was used to produce tiled images of each entire well. All dilutions and antibody neutralizations were performed in triplicate, and mean plaques per well were used to calculate the neutralization titer. The neutralization titer (given as PRNT50) of each serum sample is defined as the reciprocal of the highest test serum dilution for which the virus infectivity is reduced by 50% when compared with the mean plaque count of the control virus with no serum added. Plaque counts for all 6 serial dilutions of serum were scored to ensure that there was a dose-response.

Some further aspects are defined in the following clauses:

Clause 1: A vaccination composition, the composition comprising: a recombinant immune complex (RIC), wherein the RIC comprises: an immunoglobulin heavy chain; an epitope tag, wherein the immunoglobulin heavy chain binds the epitope tag; and at least a fragment of a herpes simplex virus type 2 (HSV2) glycoprotein D (gD).

Clause 2: The vaccination composition of Clause 1, wherein the HSV2 gD is a full-length protein.

Clause 3: The vaccination composition of Clause 1 or Clause 2, wherein the epitope tag is linked to the C-terminus of the HSV2 gD.

Clause 4: The vaccination composition of any one of the preceding Clauses 1-3, wherein the HSV2 gD is linked to the C-terminus of the immunoglobulin heavy chain.

Clause 5: The vaccination composition of any one of the preceding Clauses 1-4, wherein the RIC further comprises an immunoglobulin light chain.

Clause 6: The vaccination composition of any one of the preceding Clauses 1-5, wherein an Immunoglobulin G (IgG) comprises the immunoglobulin heavy chain.

Clause 7: The vaccination composition of any one of the preceding Clauses 1-6, wherein the IgG comprises a human or humanized IgG.

Clause 8: The vaccination composition of any one of the preceding Clauses 1-7, wherein a humanized 6D8 monoclonal antibody comprises the immunoglobulin heavy chain and wherein the epitope tag comprises a 6D8 epitope tag.

Clause 9: The vaccination composition of any one of the preceding Clauses 1-8, wherein the vaccination composition substantially cross-neutralizes herpes simplex virus type 1 (HSV1) and HSV2 when administered to a mammalian subject infected with HSV1 and HSV2.

Clause 10: A method of generating an immune response against a herpes simplex virus type 2 (HSV2) in a mammalian subject, the method comprising: administering to the mammalian subject a recombinant immune complex (RIC) that comprises: an immunoglobulin heavy chain; an epitope tag, wherein the immunoglobulin heavy chain binds the epitope tag; and at least a fragment of an HSV2 glycoprotein D (gD).

Clause 11: The method of Clause 10, wherein the HSV2 gD is a full-length protein.

Clause 12: The method of Clause 10 or Clause 11, wherein the epitope tag is linked to the C-terminus of the HSV2.

Clause 13: The method of any one of the preceding Clauses 10-12, wherein the HSV2 gD is linked to the C-terminus of the immunoglobulin heavy chain.

Clause 14: The method of any one of the preceding Clauses 10-13, wherein the RIC further comprises an immunoglobulin light chain.

Clause 15: The method of any one of the preceding Clauses 10-14, wherein an Immunoglobulin G (IgG) comprises the immunoglobulin heavy chain.

Clause 16: The method of any one of the preceding Clauses 10-15, wherein the IgG comprises a human or humanized IgG.

Clause 17: The method of any one of the preceding Clauses 10-16, wherein a humanized 6D8 monoclonal antibody comprises the immunoglobulin heavy chain and wherein the epitope tag comprises a 6D8 epitope tag.

Clause 18: The method of any one of the preceding Clauses 10-17, wherein the mammalian subject is infected with herpes simplex virus type 1 (HSV1) and HSV2, and wherein the RIC substantially cross-neutralizes the HSV1 and the HSV2 in the mammalian subject.

Clause 19: A recombinant vector comprising a nucleic acid molecule that encodes a recombinant immune complex (RIC) that comprises: an immunoglobulin heavy chain; an epitope tag, wherein the immunoglobulin heavy chain binds the epitope tag; and at least a fragment of a herpes simplex virus type 2 (HSV2) glycoprotein D (gD).

Clause 20: The recombinant vector of Clause 19, comprising a nucleic acid molecule that encodes a recombinant immune complex (RIC) that comprises: an immunoglobulin heavy chain; an epitope tag, wherein the immunoglobulin heavy chain binds the epitope tag; and at least a fragment of a herpes simplex virus type 2 (HSV2) glycoprotein D (gD).

While the foregoing disclosure has been described in some detail by way of illustration and example for purposes of clarity and understanding, it will be clear to one of ordinary skill in the art from a reading of this disclosure that various changes in form and detail can be made without departing from the true scope of the disclosure and may be practiced within the scope of the appended claims. For example, all the methods, systems, and/or computer readable media or other aspects thereof can be used in various combinations. All patents, patent applications, websites, other publications or documents, and the like cited herein are incorporated by reference in their entirety for all purposes to the same extent as if each individual item were specifically and individually indicated to be so incorporated by reference.

What is claimed is:

1. A vaccination composition, the composition comprising: a recombinant immune complex (RIC), wherein the RIC comprises: an immunoglobulin heavy chain; an epitope tag, wherein the immunoglobulin heavy chain binds the epitope tag; and at least a fragment of a herpes simplex virus type 2 (HSV2) glycoprotein D (gD).

2. The vaccination composition of claim 1, wherein the HSV2 gD is a full-length protein.

3. The vaccination composition of claim 1, wherein the epitope tag is linked to the C-terminus of the HSV2 gD.

4. The vaccination composition of claim 1, wherein the HSV2 gD is linked to the C-terminus of the immunoglobulin heavy chain.

5. The vaccination composition of claim 1, wherein the RIC further comprises an immunoglobulin light chain.

6. The vaccination composition of claim 1, wherein an Immunoglobulin G (IgG) comprises the immunoglobulin heavy chain.

7. The vaccination composition of claim 6, wherein the IgG comprises a human or humanized IgG.

8. The vaccination composition of claim 1, wherein a humanized 6D8 monoclonal antibody comprises the immunoglobulin heavy chain and wherein the epitope tag comprises a 6D8 epitope tag.

9. The vaccination composition of claim 1, wherein the vaccination composition substantially cross-neutralizes herpes simplex virus type 1 (HSV1) and HSV2 when administered to a mammalian subject infected with HSV1 and HSV2.

10. A method of generating an immune response against a herpes simplex virus type 2 (HSV2) in a mammalian subject, the method comprising: administering to the mammalian subject a recombinant immune complex (RIC) that comprises: an immunoglobulin heavy chain; an epitope tag, wherein the immunoglobulin heavy chain binds the epitope tag; and at least a fragment of an HSV2 glycoprotein D (gD).

11. The method of claim 10, wherein the HSV2 gD is a full-length protein.

12. The method of claim 10, wherein the epitope tag is linked to the C-terminus of the HSV2.

13. The method of claim 10, wherein the HSV2 gD is linked to the C-terminus of the immunoglobulin heavy chain.

14. The method of claim 10, wherein the RIC further comprises an immunoglobulin light chain.

15. The method of claim 10, wherein an Immunoglobulin G (IgG) comprises the immunoglobulin heavy chain.

16. The method of claim 15, wherein the IgG comprises a human or humanized IgG.

17. The method of claim 10, wherein a humanized 6D8 monoclonal antibody comprises the immunoglobulin heavy chain and wherein the epitope tag comprises a 6D8 epitope tag.

18. The method of claim 10, wherein the mammalian subject is infected with herpes simplex virus type 1 (HSV1) and HSV2, and wherein the RIC substantially cross-neutralizes the HSV1 and the HSV2 in the mammalian subject.

19. A recombinant vector comprising a nucleic acid molecule that encodes a recombinant immune complex (RIC) that comprises: an immunoglobulin heavy chain; an epitope tag, wherein the immunoglobulin heavy chain binds the epitope tag; and at least a fragment of a herpes simplex virus type 2 (HSV2) glycoprotein D (gD).

20. The recombinant vector of claim 19, wherein a plasmid comprises the recombinant vector.


SEQUENCE LISTING (16 sequences)

SEQ ID NO: 1 - DNA, 57 bp, synthetic construct
  misc_feature 1..57: note = Synthetic: IgG heavy chain signal peptide
  source 1..57: mol_type = other DNA
atgggatggtcttgcatcatactctttcttgttgcaactgctacaggtgtccactct

SEQ ID NO: 2 - DNA, 1338 bp, synthetic construct
  misc_feature 1..1338: note = Synthetic: IgG1 heavy chain
  source 1..1338: mol_type = other DNA
gatgttcagcttctcgagtctggaggtggtcttgtgcaacctggaggttccttgagactctcctgtgcagcttcagggtttgacttcagtaggtactggatgagttgggttcgtcaagctcctgggaaaggactagaatggattggagagatcaatccagattcaagtaccatcaactatactccatctctgaaggatcgcttcaccatttccagagacaatgccaagaacacgttgtatcttcagatgaacagcttgaggactgaagacacagccttgtactactgcacaagacagggctatggctacaactactggggtcaaggcaccactgtcacagtgtcttcagctagcaccaaaggtccatcggtctttccactggcaccttcttccaagagtacttctggaggcacagctgcactgggttgtcttgtcaaggactactttccagaacctgttacggtttcgtggaactcaggtgctctgaccagtggagtgcacacctttccagctgttcttcagtcctcaggattgtattctcttagcagtgttgtgactgttccatcctcaagcttgggcactcagacctacatctgcaatgtgaatcacaaacccagcaacaccaaggttgacaagaaagttgagcccaagtcttgtgacaagactcatacgtgtccaccgtgcccagcacctgaacttcttggaggaccgtcagtcttcttgtttcctccaaagcctaaggataccttgatgatctccaggactcctgaagtcacatgtgtagttgtggatgtgagccatgaagatcctgaggtgaagttcaactggtatgtggatggtgtggaagtgcacaatgccaagacaaagccgagagaggaacagtacaacagcacgtacagggttgtctcagttctcactgttctccatcaagattggttgaatggcaaagagtacaagtgcaaggtctccaacaaagccctcccagcccccattgagaagaccatttccaaagcgaaagggcaaccccgtgaaccacaagtgtacacacttcctccatctcgcgatgaactgaccaagaaccaggtcagcttgacttgcctggtgaaaggcttctatccctctgacatagctgtagagtgggagagcaatgggcaaccggagaacaactacaagactacacctcccgttctcgattctgacggctccttcttcctctacagcaagctcacagtggacaagagcaggtggcaacaagggaatgtcttctcatgctccgtgatgcatgaggctcttcacaatcactacacacagaagagtctctccttgtctccgggtaaa

SEQ ID NO: 3 - DNA, 45 bp, synthetic construct
  misc_feature 1..45: note = Synthetic: Linker
  source 1..45: mol_type = other DNA
ggaggtggcggatcaggtggaggcggttcaggcggaggtggatcc

SEQ ID NO: 4 - DNA, 918 bp, synthetic construct
  misc_feature 1..918: note = Synthetic: HSV gD306
  source 1..918: mol_type = other DNA
aaatatgcattagctgatcctagtcttaaaatggcagatcctaatagattcagaggaaagaatcttccagttttggaccaacttacagatcctccaggagtcaaaagagtttatcatattcaaccttctcttgaggaccctttccaacctcctagcatacctataactgtttattacgctgtgcttgaaagggcttgtaggtctgtgctcctacatgcaccatctgaggctcctcagatagttagaggtgcttctgatgaagcaaggaaacatacttacaatttgaccattgcttggtataggatgggagacaattgtgctatccctattaccgttatggagtacactgaatgtccctacaacaagtctttgggagtttgccctattagaactcagccaaggtggagttactatgattccttctctgcagtttcagaagataatttgggattcttgatgcatgctcctgcctttgagacagctggcacatacttgaggcttgtgaagattaacgactggactgagattacacaattcatacttgaacacagggccagagcatcctgcaagtatgctctcccacttaggattcctccagctgcatgcctcacctcaaaagcttatcaacagggtgtgactgtcgatagtattggcatgttacctcgttttatcccagagaaccagagaaccgttgcattgtactcacttaagatcgcaggatggcacggtccaaagcctccatacacttcaactttgcttccacctgagttgtctgacacaactaacgctacacaacctgaactcgttccagaagacccagaagattctgccctcttagaagatccagctggcactgtgtcttcacaaattcctccaaactggcacattccatcaattcaggatgttgctccacaccat

SEQ ID NO: 5 - skipped

SEQ ID NO: 6 - DNA, 45 bp, synthetic construct
  misc_feature 1..45: note = Synthetic: Epitope tag
  source 1..45: mol_type = other DNA
cataacactcctgtttacaagctggacatatctgaggcaactcaa

SEQ ID NO: 7 - DNA, 57 bp, synthetic construct
  misc_feature 1..57: note = Synthetic: IgG light chain signal peptide
  source 1..57: mol_type = other DNA
atgggatggtcttgcatcattctcttcttggtagccacagctacaggtgtccactcc

SEQ ID NO: 8 - DNA, 654 bp, synthetic construct
  misc_feature 1..654: note = Synthetic: IgG light chain
  source 1..654: mol_type = other DNA
gatgttttgatgactcaaagccctctctcacttcctgtgactcttggacagcccgcatccatatcttgcagatctagtcagagtattgttcatagtaacggcaacacctacttggaatggtatctgcagaaaccaggccagtctccaaagcttctgatctacaaggcttccaatcgtttctctggtgtcccagacaggtttagtggcagtggatcagggactgacttcacattgaagatcagcagagttgaggctgaagatgcgggagtgtactattgtcttcaaggttcacatgttccgtcaacgtttggaggtgggaccaaagtggagatcaagactgttgcggcgccatctgtcttcatctttcctccatctgatgaacaactcaagtctggaactgcttctgttgtgtgccttctgaacaacttctatcctagagaagccaaagtacagtggaaggttgacaatgctcttcaatcaggtaactcccaggagagtgtcacagagcaagattccaaggattccacctacagcctctcaagtaccttgacgttgagcaaggcagactatgagaaacacaaagtgtacgcatgcgaagtcactcatcagggcctgtcatcacccgtgacaaagagcttcaacaggggagagtgt

SEQ ID NO: 9 - protein, 19 aa, synthetic construct
  REGION 1..19: note = Synthetic: IgG heavy chain signal peptide
MGWSCIILFLVATATGVHS

SEQ ID NO: 10 - protein, 446 aa, synthetic construct
  REGION 1..446: note = Synthetic: IgG heavy chain
DVQLLESGGGLVQPGGSLRLSCAASGFDFSRYWMSWVRQAPGKGLEWIGEINPDSSTINYTPSLKDRFTISRDNAKNTLYLQMNSLRTEDTALYYCTRQGYGYNYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 11 - protein, 15 aa, synthetic construct
  REGION 1..15: note = Synthetic: Linker
GGGGSGGGGSGGGGS

SEQ ID NO: 12 - protein, 306 aa, synthetic construct
  REGION 1..306: note = Synthetic: HSV gD306
KYALADPSLKMADPNRFRGKNLPVLDQLTDPPGVKRVYHIQPSLEDPFQPPSIPITVYYAVLERACRSVLLHAPSEAPQIVRGASDEARKHTYNLTIAWYRMGDNCAIPITVMEYTECPYNKSLGVCPIRTQPRWSYYDSFSAVSEDNLGFLMHAPAFETAGTYLRLVKINDWTEITQFILEHRARASCKYALPLRIPPAACLTSKAYQQGVTVDSIGMLPRFIPENQRTVALYSLKIAGWHGPKPPYTSTLLPPELSDTTNATQPELVPEDPEDSALLEDPAGTVSSQIPPNWHIPSIQDVAPHH

SEQ ID NO: 13 - skipped

SEQ ID NO: 14 - protein, 15 aa, synthetic construct
  REGION 1..15: note = Synthetic: Epitope tag
HNTPVYKLDISEATQ

SEQ ID NO: 15 - DNA, 33 bp, synthetic construct
  misc_feature 1..33: note = Synthetic: gD-Bam-F primer
  source 1..33: mol_type = other DNA
ggggatccaaatatgcattagctgatcctagtc

SEQ ID NO: 16 - DNA, 27 bp, synthetic construct
  misc_feature 1..27: note = Synthetic: gD306-Spe-R primer
  source 1..27: mol_type = other DNA
gcaactagtatggtgtggagcaacatc